(19) United States
(12) Patent Application Publication
(10) Pub. No.: US 2014/0039450 A1
(43) Pub. Date: Feb. 6, 2014

(54) Method and Apparatus for Treating Respiratory Disease

(71) Applicant: P. A. Handford, Oxon (GB)

(72) Inventors: Alexander L. Green, Oxford (GB); Tipu Aziz, Oxford (GB); Robert Davies, Lower Heyford (GB); Jonathan Hyam, Oxford (GB)

(73) Assignee: Isis Innovation Ltd., Summertown, Oxford (GB)

(21) Appl. No.: 13/878,389

(22) PCT Filed: Oct. 6, 2011
(86) PCT No.: PCT/GB11/51924

§ 371 Date: Oct. 25, 2013

(30) Foreign Application Priority Data
Oct. 6, 2010 (GB) 1016812.8

(51) Int. Cl. A61N 1/36; A61N 1/05; A61M 5/172

(52) U.S. Cl. CPC A61N 1/3601; A61M 5/1723; A61N 1/0551
USPC 604/503; 604/500; 607/116; 607/42

(57) ABSTRACT

A method of influencing bronchoconstriction in a mammal comprising applying a stimulation in one or more regions of the brain of the mammal, and an apparatus therefore. The method and apparatus may be used to treat a respiratory disease or sleep apnea.

### FIELD OF THE INVENTION

The present invention relates to the treatment of respiratory disease by deep brain stimulation.

### BACKGROUND OF THE INVENTION

Deep Brain Stimulation (DBS) is a surgical procedure used to treat a variety of disabling neurological symptoms—most commonly the debilitating symptoms of Parkinson's disease (PD), such as tremor, rigidity, stiffness, slowed movement, and walking problems. The procedure is also used to treat other conditions such as dystonia, chronic pain and depression. DBS uses a surgically implanted, battery-operated neurostimulator to deliver electrical stimulation to targeted areas in the brain. In PD patients, this stimulation to targeted areas in the brain that control movement and blocks the abnormal nerve signals that cause tremor and PD symptoms. Generally, these targets are the thalamus, subthalamic nucleus, and globus pallidus.

The DBS system consists of three components: the lead, the extension, and the neurostimulator. The lead (or electrode)—a thin, insulated wire—is inserted through a small opening in the skull and implanted in the brain. The tip of the electrode is positioned within the targeted brain area. The extension is an insulated wire that is passed under the skin of the head, neck, and shoulder, connecting the lead to the neurostimulator. The neurostimulator (the ‘battery pack’) is the third component and is usually implanted under the skin near the collarbone or lower in the chest or under the skin over the abdomen. Once the system is in place, electrical impulses are sent from the neurostimulator along the extension wire and the lead and into the brain.

Physiological studies in humans have demonstrated that the PAG, subthalamic nucleus (STN) and pedunculopontine nucleus (PPN) can modulate parameters recognised to be under autonomic control. For example, stimulation of the STN has been shown to elevate heart rate and arterial blood pressure, regulate sweating and to resist the postural blood pressure fall with head-up tilt (Thornton, J Physiology 2002; 539(2):615-621, Trachani, Clinical Neurology Neurosurgery 2009; E-publication). PAG stimulation has been shown to reduce or elevate systolic blood pressure by 14 mmHg and 16 mmHg, respectively, and resist the postural blood pressure drop on standing (Green, Neuroreport 2005; 16(16):1741-1745, Green, Experimental Physiology 2006; 93(9):102-1028). The PPN lies within the mesencephalic locomotor region (Mogenson, Brain Research 1989; 485:396-398, Skinner, Neuroreport 1990; 1:183-186 and Neuroreport 1990; 1:207-210). When stimulated, this nucleus causes heart rate and arterial blood pressure elevation in decerebrate or anaesthetised animals even after muscle paralysis (Bedford, J Applied Physiology 1992; 72:121-127, Chong, European J Physiology 1997; 434:280-284).

Respiratory disease is a major health concern for humans and a common cause of illness and death. Respiratory diseases affect the bronchus and lungs, and include diseases such as chronic obstructive pulmonary disease (COPD), bronchial asthma, lung cancer and bronchial adenoma. Bronchoconstriction is a crucial component underpinning the pathologies of asthma and chronic obstructive pulmonary disease. Treatment of respiratory diseases may involve medication, often administered via inhalation, for example bronchodilators, corticosteroids, antibiotics and anticoagulants. For example, drugs currently used in COPD may be largely classified into corticosteroids, bronchodilators, and combined therapy. Corticosteroids are used for COPD patients with severe or recurrent symptoms, and prolonged dosage is not recommended because side effects such as muscular weakness, functional reduction, and respiratory failure are caused by the agents. Bronchodilators may be sub-classified into beta-2 agonists, anticholinergics, and methylxanthines. Beta-2 agonists induce relaxation of airway smooth muscle, may be sub-classified into fast-acting and slow-acting drugs, and have side effects such as tachycardia, tremor, hypokalemia, and tachyphylaxis. Treatment of respiratory diseases may also include physiotherapy or vaccination.

Sleep apnea is a sleep disorder characterized by pauses in breathing during sleep. There are three distinct forms of sleep apnea: central, obstructive, and complex (i.e., a combination of central and obstructive). In central sleep apnea breathing is interrupted by the lack of respiratory effort; in obstructive sleep apnea breathing is interrupted by a physical block to airflow despite respiratory effort. Upper airway increased muscle tone and obstruction is a feature of obstructive sleep apnea in addition to autonomic and respiratory deficiencies in standard autonomic tests. Chronic severe obstructive sleep apnea requires treatment to prevent low blood oxygen (hypoxemia), sleep deprivation, and other complications, such as a severe form of congestive heart failure. Treatment may include lifestyle changes, changing sleeping position, devices to keep the airways open during sleep or surgery.

WO93/01862 and US2007/0106339 disclose methods and devices for treating bronchial constriction and respiratory disorders by providing an electrical impulse to the vagus nerve, a peripheral part of the parasympathetic nervous system (vagus nerve stimulation, VNS). However, the data provided in these applications demonstrate no or little therapeutic improvement. Furthermore, VNS for epilepsy is only partially effective and less so than DBS.

It is an object of the present invention to provide an alternative method and apparatus for treating respiratory disease and sleep apnea.

### SUMMARY OF THE INVENTION

Accordingly, according to a first aspect the invention provides a method of influencing bronchoconstriction in a mammal comprising applying a stimulation in one or more regions of the brain of the mammal. According to a second aspect the invention provides a method of treating a respiratory disease or sleep apnea in a mammal comprising applying a stimulation in one or more regions of the brain of the mammal.

This application of intracranial surgery/DBS for respiratory disease and the like is a large paradigm shift for disease that is currently managed by physicians alone, for example there is no routine surgery for asthma. Although there is a suggested pioneering surgical option for asthma that involves destroying/ablating airway smooth muscle, this is quite destructive especially when you want to protect lung tissue to maximise how much of it can contribute to gas exchange (Cox et al. New England Journal of Medicine 2007; 356(13):1327-1337). The technique described herein will preserve lung tissue in patients in whom the volume of available functioning lung parenchyma is vital to the optimisation of their respiratory function in the face of their lung disease's acute exacerbations.

A further advantage over existing drug treatments is that the inventive therapy will be administered when required without the patient necessarily having to activate it. This may be particularly important during severe bronchospasm. There is a concerning phenomenon in near-fatal asthma whereby the patient's perception of dyspnoea is blunted and therefore they under-estimate the degree of airway obstruction and the severity of the asthmatic attack. Accordingly, they do not self-administer life-saving drug therapy sufficiently in the face of potentially-fatal bronchoconstriction (Eckert Eur Respir J 2004, Barreiro Eur respir J 2004, Kikuchi New Eng J Med 1994). The inventive therapy will avoid this dangerous scenario as stimulation therapy can be continuous.

Furthermore, by targeting the central drive of respiration, the resulting effect is likely to be much more powerful than the targeting of a peripheral drive, such as VNS. VNS only targets one aspect of autonomic function, namely the vagal branch of the parasympathetic nervous system which is a peripheral nerve. The application described herein targets areas within the brain which are part of or directly modulate the complex system of reciprocally-connected parts of the central nervous system known as the central autonomic network (CAN) which is still only slowly being delineated by contemporary neuroscience. The CAN is comprised by structures throughout the neuraxis within the cerebral cortex (including the amygdala, insula and anterior cingulate cortex (ACC)), diencephalon (including the hypothalamus and thalamus), midbrain (PAG), pons (PPN, locus coeruleus (LC), parabrachial nuclei (PBN)), medulla and spinal cord. It is therefore surprising that deep brain stimulation can manipulate such an intricate central neural complex to produce such a beneficial effect on lung function.

The CAN is involved in the processing and modulation of numerous body systems including endocrine, pain and motor pathways. Influencing the function of the CAN rather than simply one of its many peripheral outflows, such as the vagus nerve, allows this application greater scope therefore to affect more body systems. Whilst VNS therapy is restricted to modulating the peripheral vagal part of the parasympathetic nervous system, the novel application described herein can modulate multiple pathways. Firstly, the CAN modulates the sympathetic nervous system. As sympathetic adrenoreceptors are found on bronchial smooth muscle and produce bronchodilation, this provides an extra source of antagonism against bronchoconstriction. Furthermore, the CAN can modulate motor function and one consequence of this is that skeletal musculature may be beneficially influenced to improve lung function. The PAG projects to medullary centres which drive the phrenic, external intercostals, internal intercostals and pelvic floor musculature which can create greater changes in intrathoracic pressure and therefore contribute to improved respiratory airflow. Another benefit of modulating the activity of parts of the CAN is that it is inextricably linked to pain pathways and the two systems have several structures in common. Such structures include the PAG and ACC which are important modifiers of the pathways which convey noxious sensations such as pain and the unpleasant feeling of dyspnoea. Improvement in discomfort associated with respiratory disease can be crucial to sufferers' quality of life.

Therefore, as the CAN itself has such a multifaceted effect on various body systems, this application can produce more varied and subtle combinations of beneficial effects for patients with respiratory diseases than simply modulating the vagal autonomic output.

These methods may be suitable to treat mammals which are suffering from a respiratory disease or sleep apnea. For example, the respiratory disease may be an obstructive lung disease, reversible airways disease, asthma, chronic obstructive pulmonary disease (COPD), emphysema, bronchitis, Ondine's curse, lung cancer, tuberculosis or a lung disease where shortness of breath is a chronic symptom.

The stimulation preferably causes bronchodilation. The stimulation is preferably deep brain stimulation. The stimulation may be achieved by applying an electrical stimulation and/or a chemical stimulation. For example, the stimulation may include at least one member selected from the group consisting of an electrical stimulation, a magnetic stimulation, an electromagnetic stimulation, a radio frequency stimulation, a biological tissue implantation, a thermal stimulation, an ultrasound stimulation and a chemical stimulation. The stimulation may include generating a voltage differential between at least two electrodes of between about −10V and about +10V with a frequency of between about 0.1 Hz and about 1 kHz, preferably between about 10 and 130 Hz, and a pulse width of 5 μsecs and 1000 μsecs.

The one or more regions of the brain may be selected from the periaqueductal grey matter of the midbrain (PAG), the subthalamic nucleus (STN), the pedunculopontine nucleus (PPN), the locus coeruleus (LC), the parabrachial nuclei (PBN), the hypothalamus, the anterior cingulate cortex (ACC), the insula cortex and the amygdala.

The method may further include feeding back a metric representative of bronchoconstriction, respiratory function including respiratory rate or blood oxygenation in an automated manner, or enabling feedback of a metric representative of bronchoconstriction, respiratory function including respiratory rate, or blood oxygenation in a manual manner, and adjusting the stimulation in response to the metric. Accordingly, advantageously the method allows chronic or on-demand activity depending on the input to the biofeedback loop (e.g. respiratory rate, pO₂).

Advantageously, this therapy can be used alone or in combination with other traditional therapies such as inhaled bronchodilators and systemic steroids.

According to further aspects the invention provides an apparatus for influencing bronchoconstriction in a mammal, comprising: a sensor detecting the extent of bronchoconstriction or derangement of respiratory activity or gas exchange in the mammal; a processor in communication with the sensor and generating a control signal based on the extent of bronchoconstriction or derangement of respiratory activity or gas exchange; a signal generator in communication with the processor generating a stimulation signal based on the control signal; and an electrode including at least two conductors in contact with a region of the brain that stimulates the region as a function of the stimulation signal in a manner influencing bronchoconstriction in the mammal.

The invention also provides an apparatus for influencing blood oxygenation in a mammal, comprising: a sensor detecting the level of oxygen in the blood of the mammal; a processor in communication with the sensor and generating a control signal based on the level of oxygen in the blood of the mammal; a signal generator in communication with the processor generating a stimulation signal based on the control signal; and an electrode including at least two conductors in contact with a region of the brain that stimulates the region as a function of the stimulation signal in a manner influencing blood oxygenation in the mammal.

The invention also provides an apparatus for stimulating a region in a human brain, comprising: a signal generator adapted to generate a signal; and at least one electrode disposed in a region of a brain in a human subject adapted to produce an output as a function of the signal to stimulate the region in a manner influencing bronchoconstriction or blood oxygenation in the human subject. The signal generator may be coupled to a receiver configured to receive stimulation parameters used for applying the stimulation by at least one member selected from the group consisting of a radio frequency signal, electrical signal, and optical signal.

Advantageously, these apparatus are active either chronically or on-demand depending on the input to the biofeedback loop (e.g. respiratory rate, pO₂).

### BRIEF DESCRIPTION OF THE DRAWINGS

FIG. 1 shows a schematic representing an instance of such a deep brain electrode stimulator system. (100=Electrode, 200=Stimulation generator±signal processor).

FIG. 2 shows a schematic of such a deep brain stimulator using feedback from a peripheral pulse oximeter which feeds back to the internal pulse generator via radiofrequency telemetry. (100=Electrode, 200=Stimulation generator±signal processor, 600=Pulse Oximeter).

FIG. 3 shows a schematic of such a deep brain stimulator using feedback from a thoracic accelerometer which feeds back to the internal pulse generator via radiofrequency telemetry. (100=Electrode, 200=Stimulation generator±signal processor, 500=Accelerometer).

FIG. 4 shows a schematic of such a deep brain stimulator using feedback from a thoracic accelerometer which feeds back to the internal pulse generator via direct cabling. (100=Electrode, 200=Stimulation generator±signal processor, 500=Accelerometer).

FIG. 5 shows a schematic of such a deep brain stimulator using feedback from a thoracic pressure gauge attached to a stretchable circumferential girdle which feeds back to the internal pulse generator via radiofrequency telemetry. (100=Electrode, 200=Stimulation generator±signal processor, 300=Thoracic girdle, 400=pressure gauge/manometer).

FIG. 6 shows a flowchart to describe a feedback mechanism to activate and de-activate stimulation based upon respiratory parameter(s).

FIG. 7 shows representative electrode locations shown on axial MRI scans (PAG=periaqueductal grey, S Thal=sensory thalamus, STN=subthalamic nucleus, PPN=pedunculopontine nucleus, GPi=globus pallidus interna).

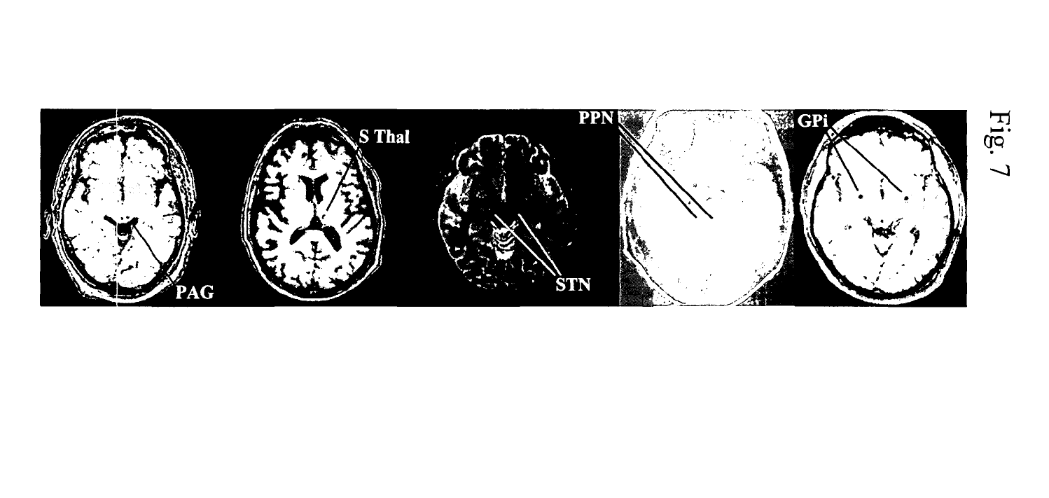

FIG. 8 shows a graph to show improvement in percentage peak expiratory flow rate with stimulation On compared to Off at each target (confidence intervals depict standard errors).

FIG. 9 shows graphs to show change in Mean PEFR within each patient On and Off stimulation of the periaqueductal grey (PAG), subthalamic nucleus (STN) and pedunculopontine nucleus (PPN).

FIG. 10 shows flow volume loops from one patient during three trials each of forced expiration with periaqueductal grey (PAG) stimulation On and Off.

FIG. 11 shows a scatterplot of Thoracic Diameter Change Ratio versus PEFR Improvement with subthalamic nucleus stimulation. Fitted regression line and confidence intervals are shown.

FIG. 12 shows a scatterplot of Thoracic Diameter Change Ratio versus PEFR Improvement with pedunculopontine stimulation. Fitted regression line and confidence intervals are shown.

FIG. 13 shows A) Sagittal MNI brain section demonstrating sites of stimulation in the pedunculopontine nucleus (PPN) group. The distribution of the PPN is shaded and overlaid on the atlas. Active electrode contacts are shown and different shades represent different patients. B) Coronal MNI brainstem section demonstrating sites of stimulation in the PPN group. C) Dorsal brainstem schematic demonstrating the PPN, locus coeruleus (LC) and lateral parabrachial nucleus (PBN) (adapted from Niewenhuys et al. 2008). SC=Superior colliculus, IC=Inferior colliculus.

FIG. 14 shows a composite table and graph depicting improvements in means of Best PEFR for each subject, Mean PEFR and Mean FEV1 in patients with stimulation of either the anterior cingulate cortex (ACC), motor thalamus or hypothalamus compared to no stimulation.

FIG. 15 shows simultaneous physiological signals in a representative patient. A) Raw LFP signal during exertional respiratory manoeuvre (microvolts); B) Time-frequency spectrogram demonstrating an increase in alpha 7-11 Hz power during maximal inspiration and forced expiration (Hz); C) Respiratory trace showing increases in thoracic circumference 5 during maximal inspiration followed by a rapid in circumference during forced expiration

### DETAILED DESCRIPTION OF THE INVENTION

The invention is based on the invasive, interventional study described herein which shows that electrical manipulation of the PAG, STN and PPN in humans has an effect on respiratory function. Specifically, it is demonstrated herein that it is possible to alter airways resistance in the human by the application of intracranial electrical stimulation and further, which specific sites of the diencephalon and brainstem confer this effect. This is important for the understanding of how the brain can control airway smooth muscle in the face of diseases such as asthma and COPD with implications for the direction of future therapies targeting the reversible components of respiratory disorders.

The invention provides a method of influencing bronchoconstriction in a mammal comprising applying a stimulation in one or more regions of the brain of the mammal. Further, the invention provides a method of treating a respiratory disease or sleep apnea in a mammal comprising applying a stimulation in one or more regions of the brain of the mammal.

Bronchoconstriction is the constriction of the airways in the lungs due to the tightening of surrounding smooth muscle, with consequent coughing, wheezing, and shortness of breath. As used herein “influencing bronchoconstriction” refers to a change (e.g., increase, decrease) in the size of the airways in the lungs of a mammal following stimulation in a region of the brain compared to the size of the airways in the lungs of the mammal before stimulation in a region of the brain. Preferably, by applying a stimulation in a region of the brain of the human, the size of the airways in the lungs of the mammal can be influenced to increase the size of the airways and therefore decrease bronchoconstriction compared to before application of the stimulation. Accordingly, preferably the stimulation causes bronchodilation. The change may be due to an inhibition of the tightening of smooth muscle surrounding the airways.

Any mammal may be treated in accordance with the methods of the invention or with the apparatus of the invention, for example dogs, cats, horses, cows, sheep and pigs. However, preferably the mammal is a human. Herein, the mammal or human to be treated may also be referred to as a subject or patient. The method of the invention is particularly useful when treating a mammal which has a respiratory disease or sleep apnea.

As used herein, “treating” means to reduce or eliminate one or more symptoms associated with the condition or disease being treated and/or to prevent or cure the condition or disease, or prevent its recurrence. The term may also encompass reducing or eliminating one or more side effects associated with a condition or disease. For example, bronchoconstriction associated with a respiratory disease may be reduced or reversed thereby allowing the subject to breathe more easily. Another example is the treatment of dyspnoea, i.e. improving the feeling of shortness of breath and reducing breathlessness. This is an enormously important symptom to control which would the improve quality of life of millions of patients with chronic lung diseases and other conditions where dyspnoea is a symptom. Dyspnoea is the one of the major forms of morbidity in all respiratory diseases. Accordingly, it is debilitating to millions of individuals worldwide who suffer from emphysema, chronic bronchitis, fibrosing alveolitis, and malignant lung diseases such as carcinoma and mesothelioma, and many others. The current treatment options are very limited and focus on improving the underlying respiratory disease however this is often not possible by current medical treatments. In fibrosing alveolitis, for example, the final stages feature marked distressing dyspnoea such that morphine pumps can be necessary to make remaining life bearable. Mesothelioma is a malignant disease of the pleura and is presently incurable. The malignant plaques progress and eventually form a non-compliant casing to restrict the changes in lung volume required for normal ventilation. The ensuing respiratory distress in the form of dyspnoea can be devastating.

A respiratory disease is any disease of the respiratory system and includes diseases of the lung, pleural cavity, bronchial tubes, trachea, upper respiratory tract and of the nerves and muscles of breathing. The invention is particularly concerned with reversible airways diseases and chronic obstructive lung diseases, such as chronic obstructive pulmonary disease (COPD) and asthma, and other diseases in which the bronchial tubes become narrowed making it hard to move air in and especially out of the lung. Such respiratory diseases also include bronchitis and emphysema. The invention is also concerned in particular with respiratory diseases which are caused by a failure of neural or autonomic control of breathing, such as Ondine's curse. The invention is further concerned with chronic lung diseases and restrictive lung diseases, such as lung cancer, tuberculosis and other lung diseases where shortness of breath is a chronic symptom.

Sleep apnea is a sleep disorder characterized by pauses in breathing during sleep. The invention is concerned with both obstructive sleep apnea and central sleep apnea. One form of central sleep apnea is Ondine's curse (also called congenital central hypoventilation syndrome (CCHS) or primary alveolar hypoventilation), which is a respiratory disorder caused by an inborn failure of autonomic control of breathing. Afflicted persons afflicted classically suffer from respiratory arrest during sleep.

By “treating sleep apnea” it is meant that the symptoms of the disease are reduced or eliminated. For example, patients may realise an increase in undisturbed sleep duration achieved, less snoring and/or reduced incidence of apnoeic attacks.

The method of influencing bronchoconstriction or treating a respiratory disease or sleep apnea in a mammal may comprise applying a stimulation in one or more regions of the brain of the mammal, for example by generating an electrical signal and/or by discharging a pharmaceutical into the one or more regions of the brain. The pharmaceutical may be selected from an inhibitory neurotransmitter agonist, an excitatory neurotransmitter antagonist, an agent that increases the level of an inhibitory neurotransmitter, an agent that decrease the level of an excitatory neurotransmitter, and a local anesthetic agent.

Thus, the brain may be stimulated in any manner known to the skilled person to achieve the desired effect. The stimulation can include at least one member selected from the group consisting of an electrical stimulation, a magnetic stimulation, an electromagnetic stimulation, a radiofrequency stimulation, a biological tissue implantation (e.g. implantation of stem cells), a thermal stimulation, an ultrasound stimulation and a chemical stimulation. Preferably the stimulation is deep brain stimulation. Deep brain stimulation is a technique which is well known to the skilled person.

As discussed, the stimulation may include the contemporary in-dwelling deep brain macroelectrode but other neuromodulation techniques are equally applicable, including gene therapies such as optogenetics whereby specific neuronal populations can be inhibited or activated from moment-to-moment by exposure to different wavelengths of light as described by Henderson (Neurosurgery 2009; 64:796-804) or by selectively-binding drug therapies. In addition, it is possible to use chemical stimulation such as targeted delivery of chemical or neurotrophic growth factor agents to brain areas transiently or chronically as described by Gill et al. (Nature Medicine 2003; 9(5):589-595); magnetic stimulation using internal probes or external fields; ultrasound using internal probes or external fields; transplantation of cells including stem cells and thermal or radiofrequency stimulation which may stimulate or lesion brain tissue. It is envisioned that these different methods of stimulation may be performed independently or in combination with one another. For example, chemical stimulation or pharmaceutical infusion may be performed independently of electrical stimulation and/or in combination with electrical stimulation.

In accordance with the invention the brain is stimulated in one or more regions. The one or more regions can include the subcallosal area, subgenual cingulate area, diencephalon (including the hypothalamus and thalamus), orbital frontal cortex, anterior insula, medial frontal cortex, dorsolateral prefrontal, dorsal anterior cortex, posterior cingulate area, premotor, orbital frontal, parietal region, ventrolateral prefrontal, dorsal cingulate, anterior cingulate cortex (ACC), caudate nucleus, anterior thalamus, nucleus accumbens; periaqueductal gray area of the midbrain (PAG), medulla, spinal cord, brainstem, and/or the surrounding or adjacent white matter tracts leading to or from the all of these listed areas or white matter tracts that are contiguous. Preferably the region includes all or part of the CAN. Thus, stimulation of any of the above brain tissue areas, as well as any white matter tracts afferent to or efferent from the abovementioned brain tissue can result in alterations or changes that alleviate or improve the cognitive impairment and/or disorder of the subject. Most preferably the brain is selectively stimulated in one or more regions selected from the periaqueductal grey matter of the midbrain (PAG), the subthalamic nucleus (STN), the pedunculopontine nucleus (PPN), the locus coeruleus (LC), the parabrachial nuclei (PBN), the hypothalamus, the ACC, the insula and the amygdala.

The stimulation parameters, for example the voltage, pulse width, frequency and electrode contacts, may be varied by the skilled person to obtain the desired results. Treatment regimens may vary and often depend on the health and age of the patient and the type and severity of the disease to be treated. Thus, the voltage may preferably range from about −10y to about +10V, most preferably about 0.5V to about 6V, or about 2V to about 4V. The pulse width may preferably range from about 20 μsec to about 20 msec, most preferably about 560 μsec to about 500 μsec, or about 90 μsec to about 200 μsec. The frequency may preferably range from about 1 Hz to about 1 kHz, most preferably about 10 Hz to about 300 Hz, or about 30 Hz to about 180 Hz, or about 90 Hz to about 130 Hz. Electrode contacts will vary from patient-to-patient. Monopolar electrical stimulation or bipolar electrical stimulation may be applied using any combination of electrode contacts. It is desired to modulate neuronal activity in the specified region of the brain, which may include the positive or negative regulation of neuronal activity, e.g. increase, decrease, masking, altering, overriding or restoring neuronal activity. Such modulation of neuronal activity may affect the degree of bronchoconstriction of a subject, allow the subject to breathe more easily or reduce breathlessness.

The methods described herein may further include feeding back a metric representative of bronchoconstriction or blood oxygenation in an automated manner, or enabling feedback of a metric representative of bronchoconstriction or blood oxygenation in a manual manner, and adjusting the stimulation in response to the metric.

In another aspect the invention provides an apparatus for influencing bronchoconstriction in a mammal, comprising: a sensor detecting the extent of bronchoconstriction or derangement of respiratory parameters (respiratory activity or gas exchange) in the mammal; a processor in communication with the sensor and generating a control signal based on the extent of bronchoconstriction or derangement of respiratory parameters; a signal generator in communication with the processor generating a stimulation signal based on the control signal; and an electrode including at least two conductors in contact with a region of the brain that stimulates the region as a function of the stimulation signal in a manner influencing bronchoconstriction in the mammal.

Some or all of this apparatus may be surgically implanted in communication with one or more regions of the brain. For example, an electrode may be implanted in communication with one or more regions of the brain, together with a signal generator and processor. The apparatus is operated to stimulate the region(s) of the brain thereby influencing bronchoconstriction or treating the respiratory disease. As an alternative or in addition to an electrode, the apparatus may include a probe, for example, an electrode assembly (i.e., electrical stimulation lead), pharmaceutical-delivery assembly (i.e., catheters) or combinations of these (i.e., a catheter having at least one electrical stimulation lead). The signal generator may comprise a signal source (i.e., electrical signal source, chemical signal source (i.e., pharmaceutical delivery pump) or magnetic signal source). The probe may be coupled to the electrical signal source, pharmaceutical delivery pump, or both which, in turn, is operated to stimulate the predetermined treatment region. Yet further, the probe and the signal generator or source can be incorporated together, wherein the signal generator and probe are formed into a unitary or single unit, such unit may comprise, one, two or more electrodes. These devices are known in the art as microstimulators, for example, Bion® which is manufactured by Advanced Bionics Corporation.

The sensor will in general detect derangement of respiratory function including but not limited to lung function tests (peak expiratory flow rate or forced expiratory volume), blood gas levels and respiration rate. These sensors may communicate directly with the processor and/or stimulation generator by direct cabling or indirectly by methods including but not limited to radio frequency telemetry.

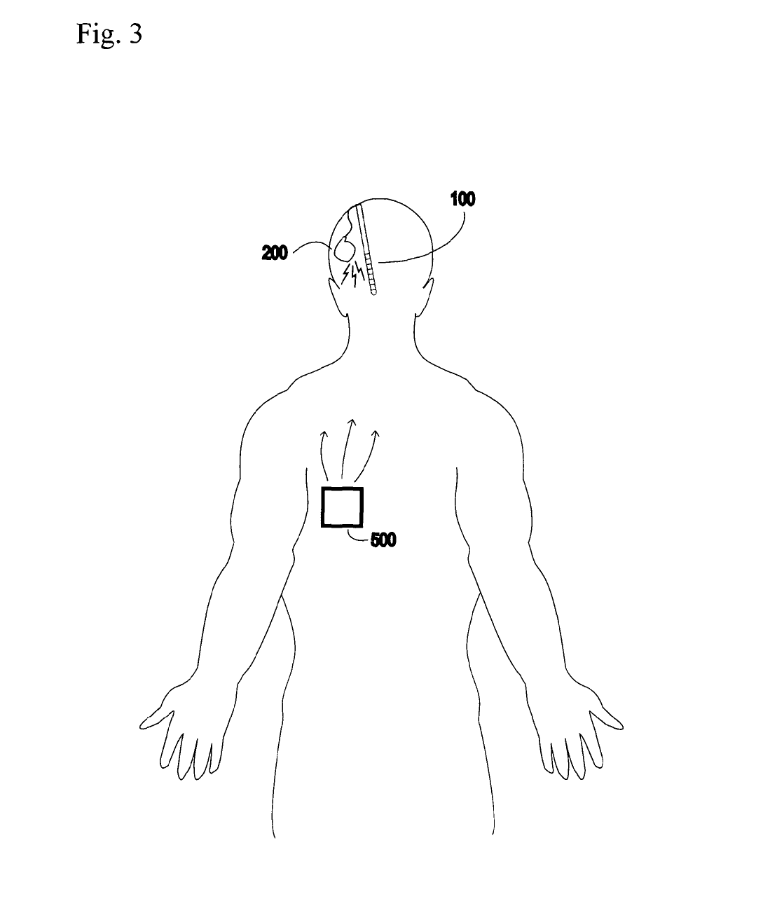

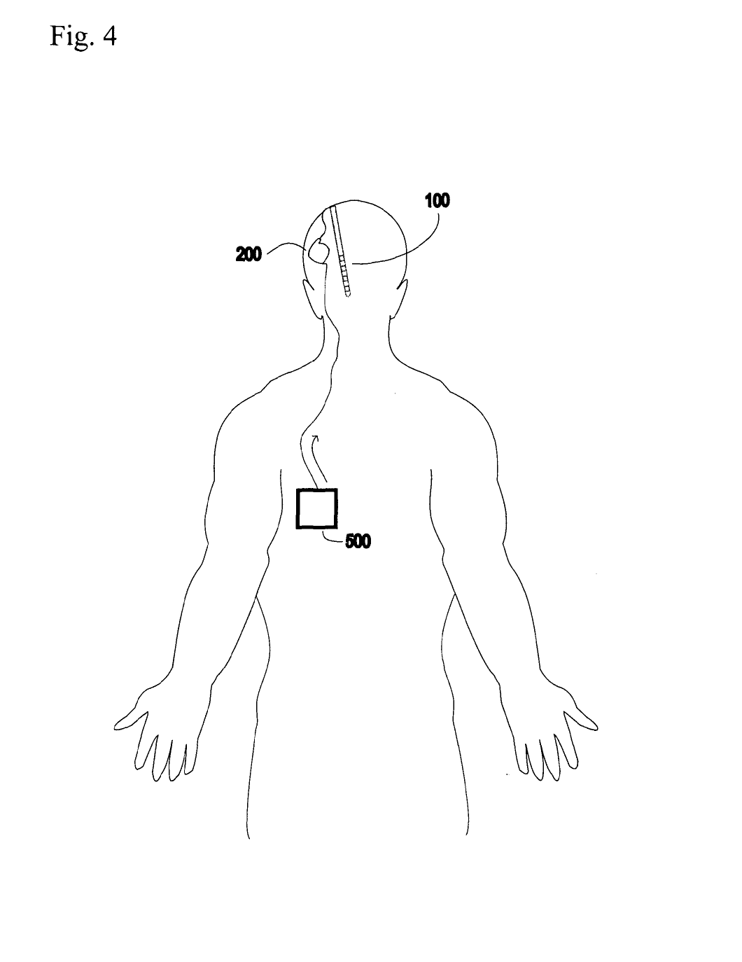

With regard to sensors which record respiratory rate or movement: One such sensor may be on the body surface or beneath the skin of the trunk whereby an accelerometer measures the continual movement of the chest with respiration which shall be detected by the processor whereby abnormally high or low rates of respiratory movement/rate detected by this sensor shall trigger the stimulation generator (FIGS. 3 and 4). FIGS. 3 and 4 show a schematic of a deep brain simulator which includes an electrode 100 and stimulation generator 200 implanted in the brain. Stimulation generator 200 may include a signal processor which is able to detect signals from an accelerometer 500 located on the body surface or beneath the skin of the trunk of the patient. In FIG. 3 the accelerometer feeds back to the internal pulse generator via the signal processor via radio frequency telemetry, whilst in FIG. 4 the accelerometer feeds back to the internal pulse generator via the signal processor via direct cabling.

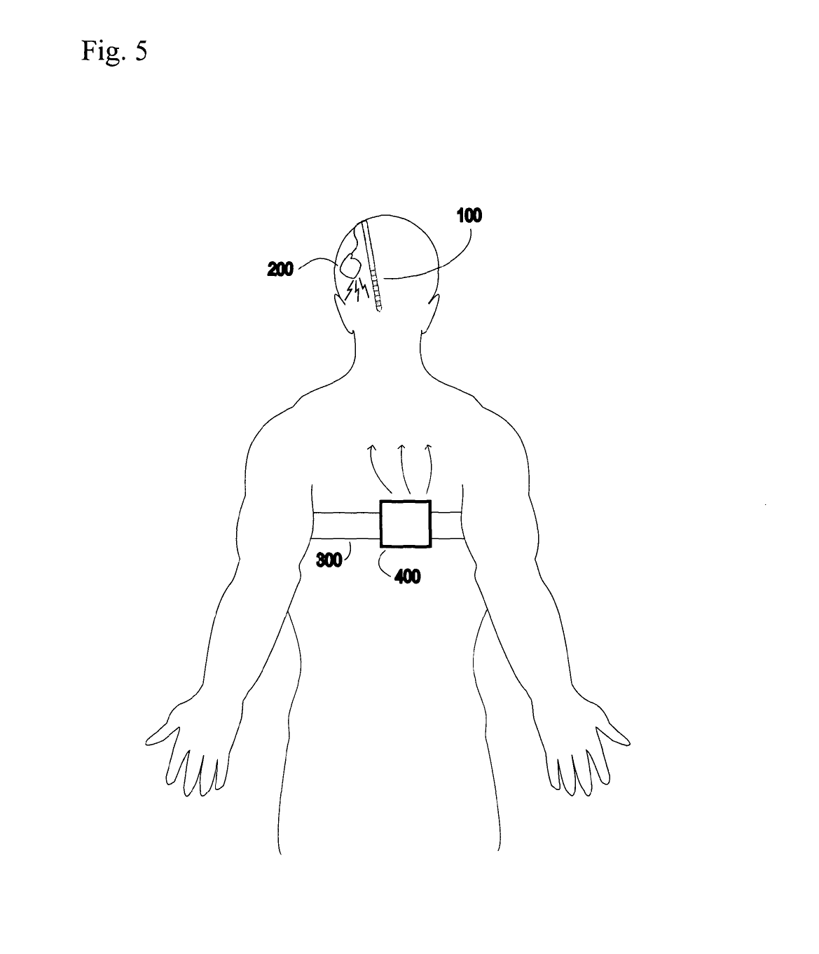

Another such sensor may be a manometer attached to a thoracic girdle applied circumferentially around the thorax which is distended by changes in thoracic volume with respiration and confers pressure changes which are sensed by the manometer (FIG. 5). The processor is triggered by abnormally high or low rates of respiratory movement/rate detected by this sensor. FIG. 5 shows a schematic of a deep brain simulator which includes an electrode 100 and stimulation generator 200 implanted in the brain. Stimulation generator 200 may include a signal processor which is able to detect signals from a thoracic pressure gauge (manometer) 400 attached to a stretachable circumferential girdle 300 located around the patient's chest.

With regard to sensors which record lung function: One such sensor may be an external spirometer measuring respiratory indices including peak expiratory flow rate and forced expiratory volume in one second. Abnormal lung function result(s) will be detected by the processor which shall then trigger the stimulator generator.

Any suitable processor may be used in accordance with the invention. Preferably the processor is a microprocessor.

Any suitable signal generator may be used in accordance with the invention. For example, the signal generator may include an implantable pulse generator (IPG), which may be available commercially or may be modified to achieve the desired results. The signal generator may include an implantable wireless receiver which is capable of receiving wireless signals from a wireless transmitter located external to the person's body. In this way a doctor, the patient, or another user may use a controller located external to the person's body to provide control signals for operation of the signal generator, for example to vary the signal parameters of electrical signals transmitted through the electrode to the region of the brain.

One of skill in the art is familiar with a variety of electrodes or electrical stimulation leads that may be utilized in the present invention. It is desirable to use an electrode or lead that contacts or conforms to the target region for optimal delivery of electrical stimulation. The electrode may be one electrode, multiple electrodes, or an array of electrodes in or around the target region. It is within the capability of the person skilled in the art to position the electrode including at least two conductors in contact with the chosen region of the brain.

In yet another aspect the invention provides an apparatus for influencing blood oxygenation in a mammal, comprising: a sensor detecting the level of oxygen in the blood of the mammal; a processor in communication with the sensor and generating a control signal based on the level of oxygen in the blood of the mammal; a signal generator in communication with the processor generating a stimulation signal based on the control signal; and an electrode including at least two conductors in contact with a region of the brain that stimulates the region as a function of the stimulation signal in a manner influencing blood oxygenation in the mammal.

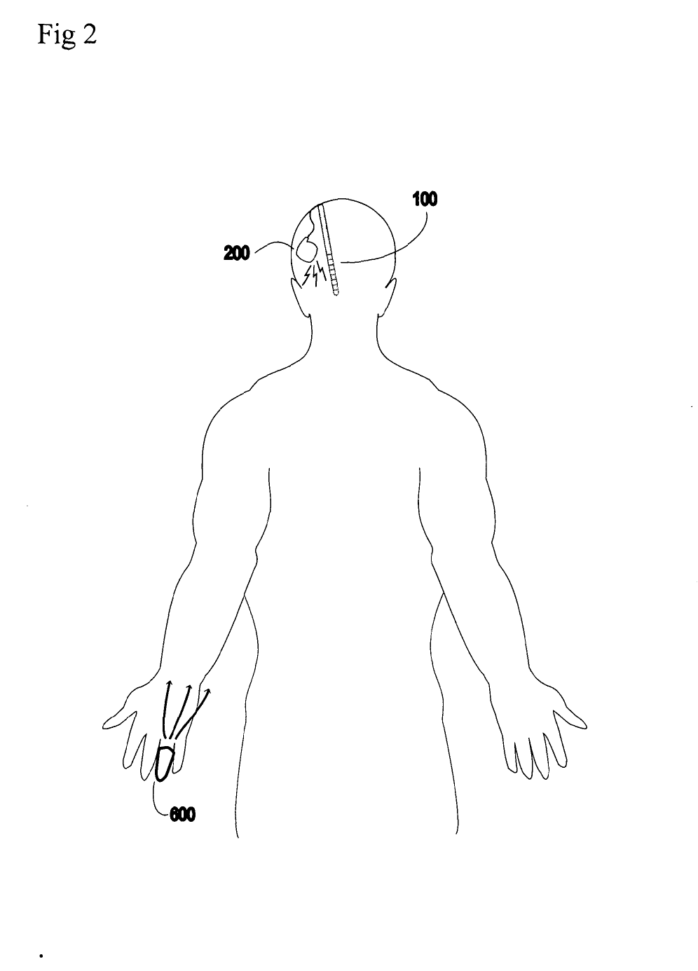

As discussed above, any suitable sensor, processor, signal generator and electrode may be selected by the skilled person in accordance with his knowledge. One such sensor for detecting the level of oxygen in the blood may be on the body surface (e.g. the finger). For example, a peripheral pulse oximeter indirectly monitors the oxygen saturation of a patient's blood which shall be detected by the processor whereby abnormally high or low levels of oxygen saturation detected by this sensor shall trigger the stimulation generator. FIG. 2 shows a schematic of such a deep brain simulator which includes an electrode 100 and stimulation generator 200 implanted in the brain. Stimulation generator 200 may include a signal processor which is able to detect signals from a pulse oximeter 600 located on the patient's fingertip.

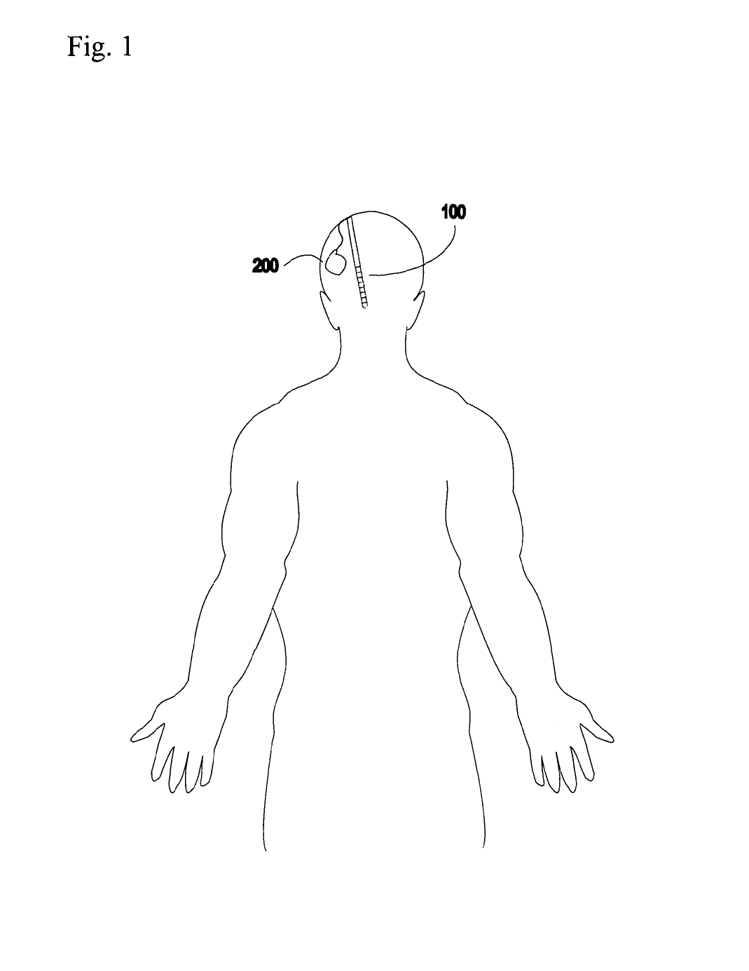

In yet another aspect the invention provides an apparatus for stimulating a region in a human brain, comprising: a signal generator adapted to generate a signal; and at least one electrode disposed in a region of a brain in a human subject adapted to produce an output as a function of the signal to stimulate the region in a manner influencing bronchoconstriction or blood oxygenation in the human subject. This apparatus is for bronchoconstriction or blood oxygenation in a human subject. For example, FIG. 1 shows a schematic of such a deep brain simulator which includes an electrode 100 and stimulation generator 200 implanted in the brain. Stimulation generator 200 may include a signal processor. Preferably the signal generator is coupled to a receiver configured to receive stimulation parameters used for applying the stimulation by at least one member selected from the group consisting of a radio frequency signal, electrical signal, and optical signal. The stimulation may be activated by the mammal or those involved in the care of the mammal if they suspect respiratory disturbance.

As discussed above, any suitable signal generator, electrode and receiver may be selected by the skilled person in accordance with his knowledge.

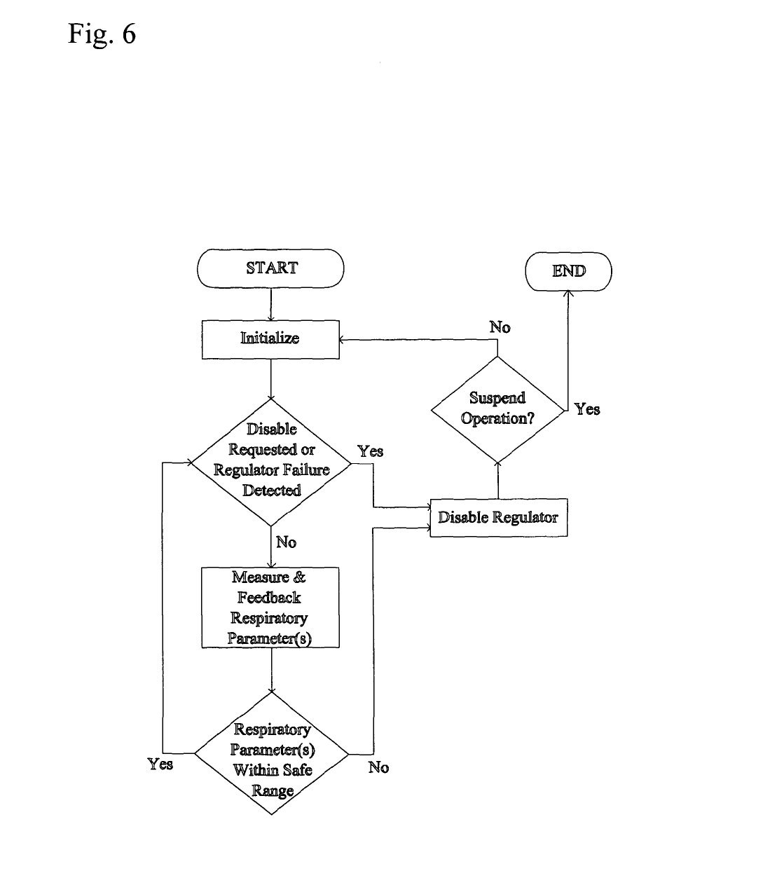

FIG. 6 is a flow diagram of a process employed by an apparatus according to the principles of the present invention. Some steps in the process may be executed in the processor and other steps maybe performed by other components or combinations of components.

The process starts and initializes to begin operation. Initialization can include any number of initialization sequences, such as power-up sequences, verifying processor operational readiness, verifying transmitters and receivers are using the same communications protocol, and so forth. The process continues by checking whether a ‘disable’ of the apparatus has been requested (e.g., manually) or an apparatus failure has been detected. An example of a failure detection maybe detection of a low power condition, loss of communications, software error, or other error that may interfere with operations of the apparatus. If disable has not been requested and failure has not been detected, the process measures and feeds back one or more respiratory parameter. In one embodiment, the respiratory parameter measurement and feedback is performed in an automated manner. In another embodiment, the respiratory parameter measurement and feedback is performed in a manual manner through use of the human-controlled feedback interface.

The process continues and determines whether the respiratory parameter is within a safe operating range, meaning that a determination is made as to whether it is safe to continue operating the apparatus. For example, if the respiratory parameter is observed to be outside a given positive or negative threshold from a nominal or normal operating pressure, the apparatus may determine that it is itself a cause of a respiratory parameter irregularity due to, for example, a failure or ‘runaway’ condition.

If the process determines it is safe to continue operating, the process may determine whether the respiratory parameter is at a desired level. If the respiratory parameter is nominal or normal, the process returns to a step of checking whether a ‘disable’ has been requested or an apparatus failure has been detected. If the process determines that the respiratory parameter is low or high, the process stimulates a region in the brain to influence a response of the respiratory parameter in the patient's body. The process thereafter continues operations.

If a ‘disable’ has been requested or a failure has been detected in the blood pressure regulator, the process disables the apparatus. Similarly, if the respiratory parameter is outside a safe operating range as described above, the process disables the apparatus. Thereafter, the process determines whether to suspend operations, optionally based on a number of criteria or as a result of the patient's triggering of a fail-safe signal (i.e., ‘disable’). If operation is not to be suspended, the process initializes the apparatus as a matter of precaution in one embodiment. If operation is to be suspended, the process ends and the apparatus is set into a safe operating mode by, for example, disabling the electrodes, powering down, or entering a ‘safe mode’. It should be understood that the process is an example embodiment used for illustration purposes only. Other embodiments within the context of regulating respiratory parameters may be employed. Some or all of the steps in the process maybe implemented in hardware, firmware, or software. If implemented in software, the software may be (i) stored locally with the processor or (ii) stored remotely and downloaded to the processor during initialization. To begin operations in a software implementation, the processor loads and executes the software in any manner known in the art.

It should be understood that any form of communications protocol(s) maybe employed to provide communications between or among the several components of the apparatus. For example, wireless communications signals may include inductive communications signals, radiofrequency (RF) communications signals, Bluetooth(R) communications signals, or other forms of wireless communications signals. For any of such wireless communications signals, various protocols can be employed, such as coding, encryption, or other protocols known to improve communications and make the device resistant to communications errors. As known in the art, communications errors may be caused by internal noise sources (e.g., low battery power, noisy amplifiers, poor analog or digital signal(s) isolation, etc.) or external noise sources, such as large electromagnetic fields (e.g., airport metal detectors, car electronics, etc.).

If chemical stimulation is used, in addition to or instead of electrical stimulation, then a drug delivery catheter may be implanted in the brain in a known manner such that the proximal end of the catheter is coupled to a pump and a discharge portion for infusing a dosage of a pharmaceutical or drug. The discharge portion of the catheter can have multiple orifices to maximize delivery of the pharmaceutical while minimizing mechanical occlusion. Any type of infusion pump can be used in the present invention, including active pumping devices, peristaltic pumps (which provide a metered amount of a drug in response to an electronic pulse generated by control circuitry associated within the device), accumulator-type pumps, drive-spring diaphragm pumps and passive pumping mechanisms (to release an agent in a constant flow or intermittently or in a bolus release).

If stimulation via transplanted cells is used, it is envisioned that the transplanted cells can replace damaged, degenerating or dead neuronal cells, deliver a biologically active molecule to the predetermined site or to ameliorate a condition and/or to enhance or stimulate existing neuronal cells. Such transplantation methods are described in U.S. Application No. US20040092010. Cells that can be transplanted can be obtained from embryonic or non-embryonic stem cells, brain biopsies, including tumour biopsies, autopsies and from animal donors.

All documents referred to herein are incorporated herein by reference in their entirety.

### EXAMPLES

The following examples are illustrative of the methods and apparatus falling within the scope of the present invention. They are not to be considered in any way limitative of the invention. Changes and modifications can be made with respect to the invention. That is, the skilled person will recognise many possible variations in these examples and can make adjustments for a variety of applications.

### Example 1

### Methods

The aim of this study was to test whether airways resistance is reduced by electrical stimulation of subcortical sites implicated in respiratory and autonomic modulation, namely the periaqueductal grey matter of the midbrain (PAG), the subthalamic nucleus (STN) and the pedunculopontine nucleus (PPN). The globus pallidus interna (GPi) and sensory thalamus are nuclei not recognised to influence autonomic performance and were used as controls.

Patients treated with deep brain stimulation for movement disorders (Parkinson's disease or dystonia) or chronic pain syndromes at the John Radcliffe Hospital, UK, and St. Andrew's Hospital, Brisbane, Australia, were recruited. All patients provided informed consent before participation in the study. Ethical permission was obtained from the Oxfordshire Research Ethics Committee C (Study No. 05/Q1605/47) and the Queensland University of Technology Human Research Ethics Committee (Study No. 0900000105) and the study conformed to the Declaration of Helsinki. Patients were excluded if they were unable to competently perform spirometry for cognitive or physical reasons in both stimulation On and Off states. The clinician overseeing subject testing was trained in the supervision of spirometry by an experienced lung function technician within the Department of Respiratory Medicine, Churchill Hospital, UK. Patients were trained to perform forced expirations as specified by the European Respiratory Society (Miller, European Respiratory J 2005; 26:319-338). Patients sat upright in a chair with the neck in a neutral position during all manoeuvres. No nose clip was applied. Values were recorded for peak expiratory flow rate (PEFR), defined as the highest flow achieved from a maximum forced expiratory manoeuvre started without hesitation from a position of maximal lung inflation (Quanjer, European Respiratory J 1997; 10 (Suppl):24, 2s-8s), and forced expiratory volume in one second (FEV1), defined as the maximal volume of air exhaled in the first second of a forced expiration from a position of full inspiration (Miller, European Respiratory J 2005; 26:319-338). Three practice forced expirations were performed to ensure patient competence in the technique. Test recordings were made during three forced expirations with stimulation on and three whilst stimulation was off. The best of the three PEFR during both on and off periods was also recorded. To allow comparison to changes in thoracic diameter, percentage PEFR improvement was also calculated.

It was decided at random whether the stimulator was on or off at the outset of the trial. After three recorded forced expirations the stimulator setting was then changed to on or off, accordingly. A period often minutes was allowed between the on and off states for the stimulation to wash-in or wash-out before the subsequent three forced expirations. Patients remained seated during this waiting period and did not partake of any food, drink or medication. A period of ten minutes was chosen as, although the motor effects of deep brain stimulation are believed to take minutes-to-hours and often longer to manifest, the reported changes in cardiorespiratory parameters such as heart rate, blood pressure and respiratory rate, are seen within seconds-to-minutes (Green, Neuroreport 2005; 16(16):1741-1745, Green, Experimental Physiology 2006; 93(9):102-1028, Green, Neuromodulation 2010, Thornton, J Physiology 2002; 539(2):615-621). In this way, as many environmental and patient factors could be kept identical between the on and off test periods. This measure also reduced the likelihood that expiratory flow changes were due to skeletal muscle/motor performance rather than airway diameter.

Patients were blinded as far as possible to the settings at which the stimulator was programmed. Patients were tested with stimulation on using parameters and electrode contacts which were currently therapeutic for their disease. Thus, the chronic pain patients were stimulated in the PAG region of the brain and the movement disorder patients were stimulated in the STN or PPN regions of the brain and patients. The globus pallidus interna (GPi) and sensory thalamus are nuclei not recognised to influence autonomic performance and were used as controls. Patients with sensory thalamus stimulation were directly comparable to PAG subjects as they both suffer from chronic pain syndromes. Several patients experienced familiar sensations when the stimulation was switched on, therefore blinding was not perfect. However, patients did not know whether stimulation was expected to be beneficial or detrimental to their lung function results.

In the movement disorder patients, change in thoracic diameter was also measured to distinguish changes in airway resistance from simply improvement in general motor function with stimulation.

### Extraparenchymal Muscle Activity Versus Airway Calibre

Applying Ohm's Law to the properties of flow along a tube,

Flow=Pressure difference between each end/Resistance

Expiratory Flow=(Pressure in Lung Parenchyma−Atmospheric Pressure)/Resistance

where, according to Poiseuille's Law, Resistance=8ηL/πr4

Therefore increases in flow can be attributable to a) increases in pressure difference between the lungs and the atmosphere and to b) increases in small airway diameter. The former is determined chiefly by thoracic and abdominal skeletal muscle and diaphragm activity to cause as great and rapid a reduction in thoracic volume to increase intrathoracic pressure. It was therefore necessary to obtain a measure of this to ensure that if peak expiratory flow rate was being improved by deep brain stimulation it was via an effect on respiratory airway diameter/resistance rather than skeletal muscle function.

To record the change in thoracic dimensions which create the pressure gradient between the lungs and the atmosphere at the mouth and nose, a pressure-sensitive thoracic girdle was fastened circumferentially around the mid-thorax at the level of the fifth rib anteriorly. Pressure changes were recorded and displayed in real time online by Spike II software and were available for subsequent analysis offline. This allowed detection of the magnitude of change in thoracic diameter during forced expiration. The ratio of thoracic diameter change (TDC—see FIG. 1) with stimulation On compared to Off was recorded as TDC ratio.

### Results

### Patients

44 patients were studied, 17 with pain syndromes and 27 with movement disorders. Within the pain syndrome group, ten patients had PAG stimulation and seven had sensory thalamus stimulation. Of the movement disorder group, ten had STN stimulation, seven had PPN stimulation and ten had GPi stimulation. Fourteen patients were female and thirty were male with a mean age of 54.7 years (SD±12.9). Patient diagnoses and stimulation parameters are summarized in Table 1. There were no cases of respiratory diseases diagnosed or requiring treatment by a respiratory physician.

TABLE 1
Summary of patient diagnoses, demographics and stimulation parameters.

| Age (yrs)/ | | Stimulator | Stimulation Parameters (Voltage, Pulse |
|---|---|---|---|
| Sex | Diagnosis | Location | Width, Frequency, Electrode Contacts) |
| 63/M | Facial Pain | PAG Unilateral | 0.5 v, 120 μsec, 15 Hz |
| 34/M | Arm Pain | PAG Unilateral | 5.8 v 120 μsec, 10 Hz |
| 45/M | Hemi-body pain | PAG Unilateral | 3.8 v, 450 μsec, 5 Hz |
| 44/F | Hemi-body pain | PAG Unilateral | 1.5 v, 180 μsec, 25 Hz |
| 70/M | Arm Pain | PAG Unilateral | 2.5 v, 120 μsec, 40 Hz |
| 63/M | Phantom limb pain | PAG Unilateral | 1.5 v, 210 μsec, 7 Hz |
| 40/F | Occipital neuralgia | PAG Unilateral | 7.3 v, 180 μsec, 15 Hz |
| 53/M | Trigeminal neuralgia | PAG Unilateral | 4.5 v, 120 μsec, 30 Hz |
| 80/M | Hemi-body pain | PAG Unilateral | 2.9 v, 450 μsec, 30 Hz |
| 61/F | Hemi-body pain | PAG Unilateral | 2.7 v, 330 μsec, 30 Hz |
| 45/M | Arm pain | SThal Unilateral | 1.2 v, 90 μsec, 40 Hz |
| 42/M | Leg pain | SThal Unilateral | 1.4 v, 90 μsec, 20 Hz |
| 32/M | Arm pain | SThal Unilateral | 0.7 v, 150 μsec, 50 Hz |
| 44/M | Hemi-body pain | SThal Unilateral | 6 v, 390 μsec, 40 Hz |
| 70/M | Arm pain | SThal Unilateral | 1.5 v, 150 μsec, 60 Hz |
| 44/F | Arm pain | SThal Unilateral | 2 v, 180 μsec, 25 Hz |
| 63/M | Facial pain | SThal Unilateral | 0.5 v, 120 μsec, 15 Hz |
| 44/M | Parkinson's Disease | STN Bilateral | 2 v, 90 μsec, 130 Hz |
| 64/M | Parkinson's Disease | STN Bilateral | 3 v, 90 μsec, 130 Hz |
| 39/M | Parkinson's Disease | STN Bilateral | Left 2 v, Right 1 v, 60 μsec, 130 Hz |
| 56/M | Parkinson's Disease | STN Bilateral | 2 v, 60 μsec, 130 Hz |
| 49/F | Parkinson's Disease | STN Bilateral | 1.5 v, 60 μsec, 130 Hz |
| 66/M | Parkinson's Disease | STN Bilateral | 1 v, 60 μsec, 130 Hz |
| 68/M | Parkinson's Disease | STN Bilateral | 1.8 v, 90 μsec, 130 Hz |
| 60/F | Parkinson's Disease | STN Bilateral | Left 2 v, Right 2.5 v, 90 μsec, 180 Hz |
| 64/M | Parkinson's Disease | STN Bilateral | Left 1.5 v Right 1.8 v, 90 μsec, 130 Hz |
| 52/F | Parkinson's Disease | STN Bilateral | 1.5 v, 60 μsec, 130 Hz |
| 47/M | Parkinson's Disease | PPN Bilateral | 2.2 v, 60 μsec, 35 Hz |
| 77/M | Parkinson's Disease | PPN Bilateral | Left 2.5 v, Right 2.8 v, 60 μsec, 35 Hz |
| 62/F | Parkinson's Disease | PPN Bilateral | 4 v, 60 μsec, 35 Hz |
| 73/M | Parkinson's Disease | PPN Bilateral | 4.3 v, 60 μsec, 35 Hz |
| 73/F | Parkinson's Disease | PPN Bilateral | 3 v, 60 μsec, 35 Hz |
| 57/M | Parkinson's Disease | PPN Bilateral | 2.2 v, 60 μsec, 20 Hz |
| 56/M | Parkinson's Disease | PPN Bilateral | 2.5 v, 60 μsec, 20 Hz |
| 59/F | Cervical Dystonia | GPi Bilateral | 2.5 v, 90 μsec, 130 Hz |
| 60/M | Segmental Dystonia | GPi Bilateral | 2.5 v, 90 μsec, 60 Hz |
| 66/M | Cervical Dystonia | GPi Bilateral | 2.5 v, 90 μsec, 90 Hz |
| 51/M | Generalized Dystonia | GPi Bilateral | 2 v, 90 μsec, 130 Hz |
| 54/M | Focal Dystonia | GPi Bilateral | 3 v, 90 μsec, 130 Hz |
| 59/F | Cervical Dystonia | GPi Bilateral | 2.5 v, 90 μsec, 130 Hz |
| 22/F | Focal Dystonia | GPi Bilateral | 2 v, 90 μsec, 130 Hz |
| 48/F | Segmental Dystonia | GPi Bilateral | 2.5 v, 90 μsec, 130 Hz |
| 35/M | Cervical Dystonia | GPi Bilateral | 2.5 v, 210 μsec, 130 Hz |
| 51/F | Cervical Dystonia | GPi Bilateral | 2 v, 90 μsec, 130 Hz |

(PAG = periaqueductal grey, SThal = sensory thalamus, STN = subthalamic nucleus, PPN = pedunculopontine nucleus, GPi = globus pallidus interna)

### Lung Function Tests: PAG and Sensory Thalamus (Control)

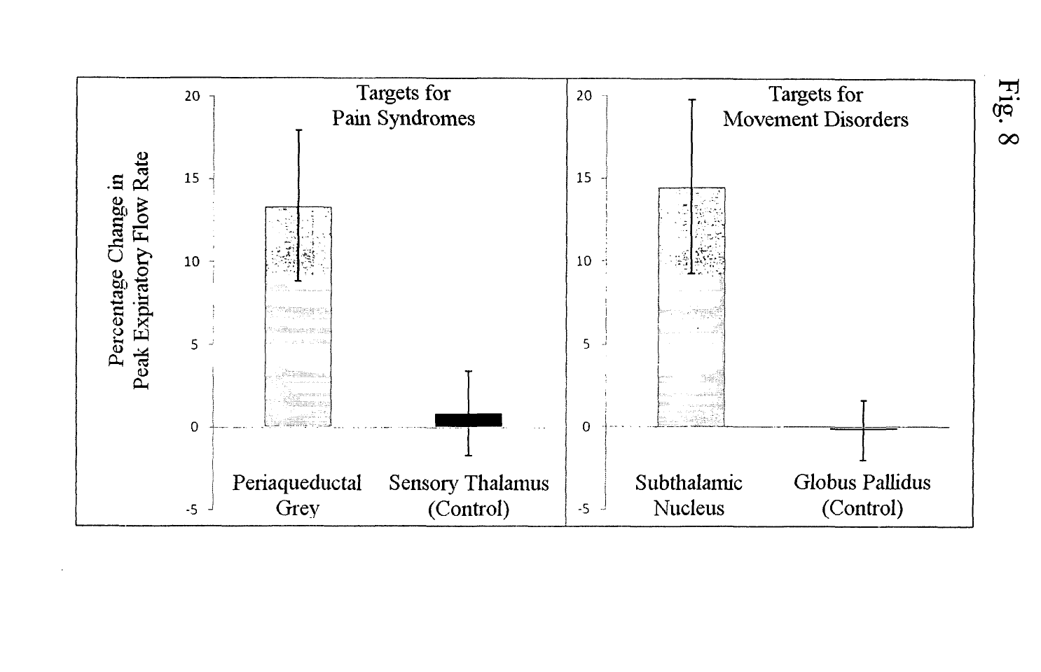

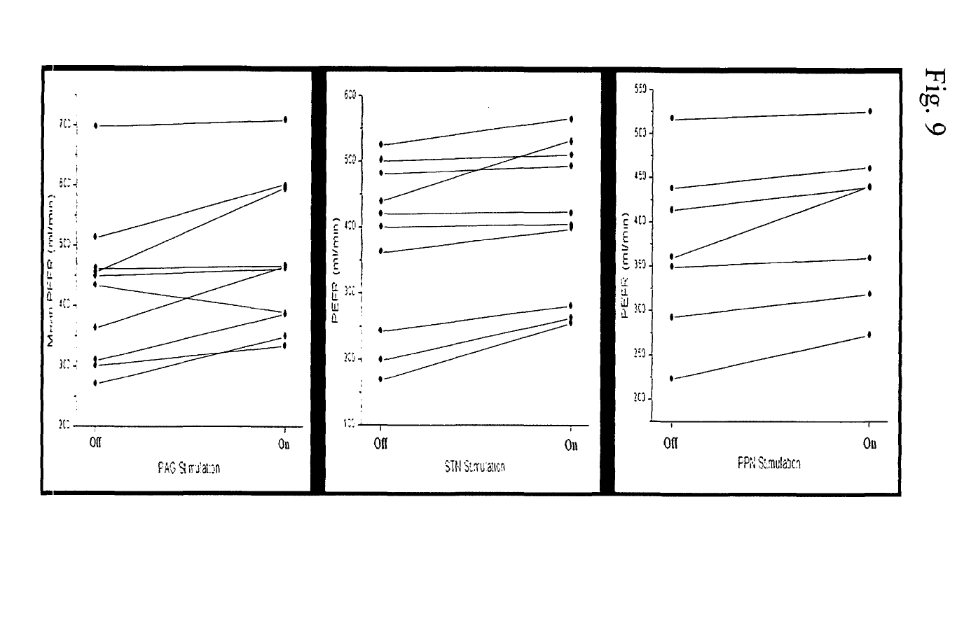

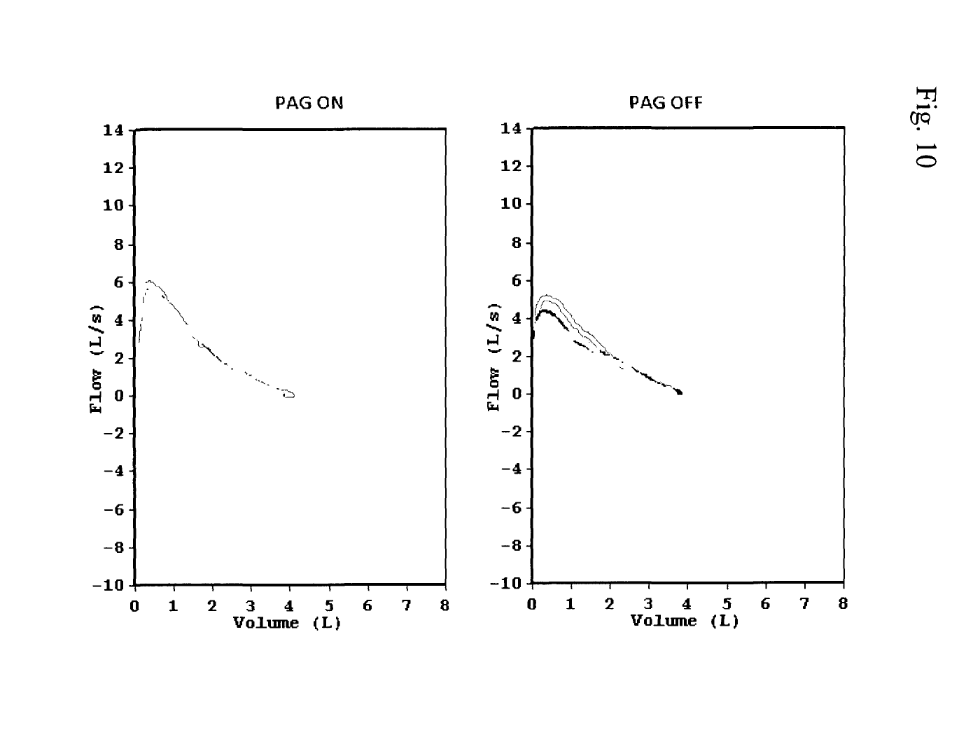

Mean PEFR percentage change was 13.4% (SE±4.6) with PAG stimulation where mean PEFR increased from 425.9 ml/min (SE±39.6) to 475.4 ml/min (SE±38.9), p=0.021. However, there was only a 0.89% (SE±2.6) mean PEFR percentage change with sensory thalamus stimulation where mean PEFR increased from 489.2 ml/min (SE±25.7) to 494.4 ml/min (SE±30.4) which was not statistically significant (p=0.667). See FIG. 8 and Table 2. Mean PEFR increased in 9 patients receiving PAG stimulation compared to the off state and decreased in one patient (see FIG. 9). An example of changes in the flow-volume loop with PAG stimulation On and Off is shown in one representative patient in FIG. 10, demonstrating the larger peak expiratory flows and the larger flow-volume area achieved with PAG stimulation. There was no significant change in mean FEV1 with stimulation of either the PAG (from mean 2.90 l/min (SE±0.27) to mean 2.92 l/min (SE±0.25), p=0.809) or sensory thalamus (from mean 3.24 l/min (SE±0.22) to mean 3.23 l/min (SE±0.25), p=0.875)).

TABLE 2.
Mean peak expiratory flow rate On versus Off stimulation within each
nucleus.

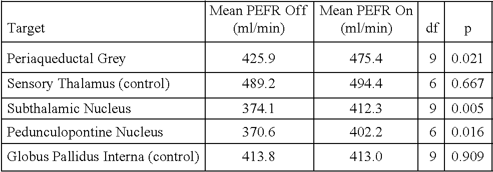

(Control groups are shaded in grey. df = degrees of freedom.)

### Lung Function Tests: STN, PPN and GPi (Control)

Mean PEFR percentage change increased by 14.5% (SE±5.3) with STN stimulation (where mean PEFR increased from 374.1 ml/min (SE±40.5) to 412.3 ml/min (SE±36.2), p=0.005) and by 9.9% (SE±3.3) with PPN stimulation (where mean PEFR increased from 370.6 ml/min (SE±36.7) to 402.2 ml/min (SE±33.5), p=0.016). However there was minimal mean PEFR percentage change of −0.2% (SE±1.8) with GPi stimulation (from a mean PEFR of 413.8 ml/min (SE±41.2) to 413.0 ml/min (SE±42.2), p=0.909). See FIG. 8 and Table 2. Mean PEFR increased in all patients receiving STN and PPN stimulation (see FIG. 9). There was no significant change in mean FEV1 with stimulation of either STN (from mean 2.32 l/min (SE±0.22) to mean 2.29 l/min (SE±0.19), p=0.776), PPN (from mean 2.55 l/min (SE±0.30) to mean 2.62 l/min (SE±0.32), p=0.411) or GPi (from mean 2.55 l/min (SE±0.28) to mean 2.55 l/min (SE±0.29), p=0.965).

There was no significant change in mean FEV1 with stimulation of either the PAG (from mean 2.90 l/min (SE±0.27) to mean 2.92 l/min (SE±0.25), p=0.809) or sensory thalamus (from mean 3.24 l/min (SE±0.22) to mean 3.23 l/min (SE±0.25), p=0.875).

### Thoracic Diameter Change v PEFR Improvement

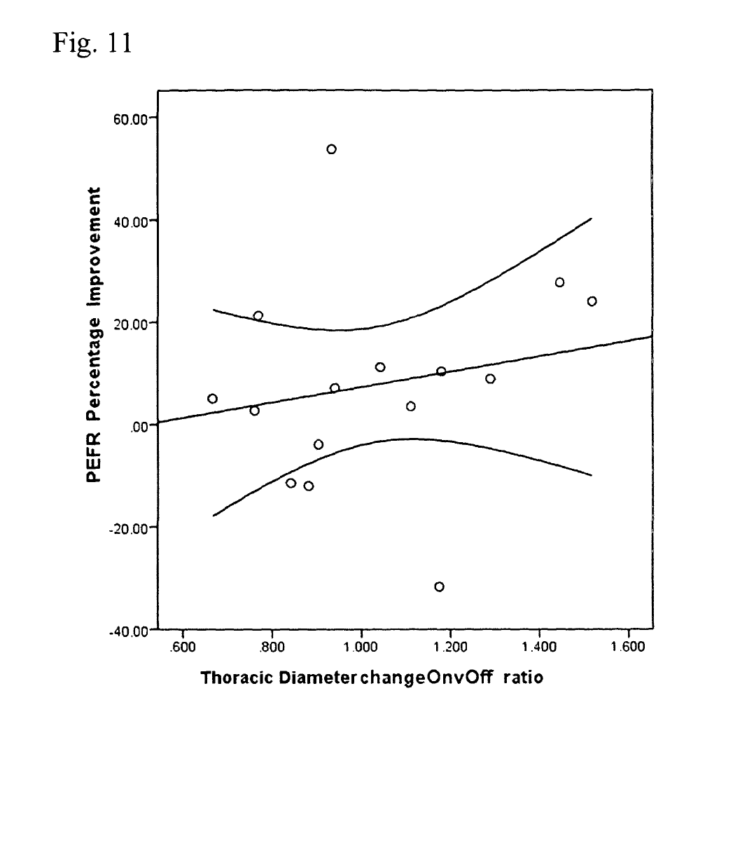

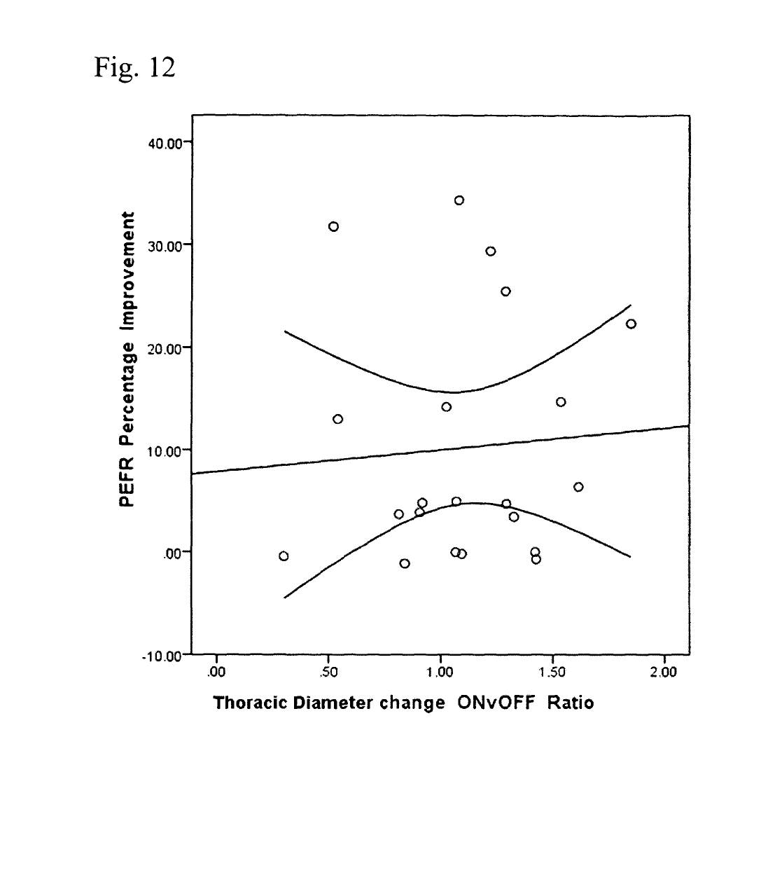

To distinguish whether the significant PEFR improvement with PPN and STN stimulation in Parkinson's Disease patients was attributable to thoracic musculoskeletal performance improvements rather than respiratory airways dilatation, TDC ratio was calculated within these two groups (seven PPN patients and five STN patients). Mean On:Off TDC ratio was 1.03 (+/−SD 0.25) and 1.1 (+/−SD 0.38) in STN and PPN groups, respectively. PEFR percentage improvement was 7.8% (+/−SD 19.80) and 10.23% (+/−SD 11.72) in these STN and PPN groups, respectively. TDC ratio and PEFR percentage improvement were poorly correlated where r=0.192, p=0.493, n=15 in the STN group (see Table 3 and FIGS. 11) and r=0.069, p=0.766, n=21 in the PPN group (see Table 3 and FIG. 12). Therefore TDC ratio only explained 3.7% and 0.5% of the variance of PEFR improvement in STN and PPN groups, respectively.

TABLE 3
Table to show results of Pearson's correlation for TDC ratio and
Percentage PEFR improvement.

TDC Ratio v PEFR
Percentage
Improvement

| | STN | PPN |
|---|---|---|
| Pearson's Correlation Coefficient r | 0.192 | 0.069 |
| Percentage of Variance | 3.7% | 0.5% |
| p value | 0.493 | 0.766 |

### Discussion

This is the first study to link the human PAG, STN and PPN to direct effects on lung function. Stimulation at all three of these autonomically-implicated deep brain areas produced a significant increase in PEFR. Within the pain group, sensory thalamus stimulation was used as a control and conferred no change in lung function. Therefore the improvement with PAG stimulation cannot be explained by a simple improvement in the patients' pain state which could have allowed them to perform the test more effectively. Within the movement disorder group, GPi stimulation did not change lung function. This, combined with the fact that the effect of STN and PPN stimulation on PEFR poorly correlated with thoracic diameter change, suggests that their effect on lung function was not simply a result of improving general skeletal motor performance. Therefore the results support a mechanism in which stimulation of these nuclei relaxes respiratory airway smooth muscle.

Although there were significant changes in PEFR in the experimental groups, there was no change in FEV1 with stimulation. This variable response in different indices of lung function is not surprising in this patient group since none suffered from respiratory disease. Consequently, the capacity for lung function change is limited in patients with near-normal airway function and calibre so variability in results between different indices is to be expected. Further studies in subjects with abnormal airway calibre and established chronic lung disease are required to more fully understand this aspect of the results identified here.

This study provides further evidence to support the putative circuitry whereby the GPi, STN and PPN are linked. The GPi and STN are proposed to project to the PPN but whereas the STN is excitatory to the PPN via glutaminergic transmission, the GPi is inhibitory both to the PPN and STN via GABAnergic transmission (Hamani, Brain 2004; 127:4-20, Jenkinson, Neuroreport. 2006; 17(6):639-41). Both STN and PPN stimulation produced a significant improvement in PEFR. Stimulation of the GPi would therefore be expected to antagonise the effect of the PPN and STN on airway resistance which is indeed seen in our results whereby there was no change in lung function with GPi stimulation.

This is the first time it has been possible to directly link the STN to the human respiratory system. The STN has been implicated impirically as a component of the respiratory network. Due to its role in inhibiting initiated responses in stop-signal paradigms, the STN is suspected to be active during breath-holding; however neuro-imaging has failed to detect any evidence of this. In this study, stimulation of the STN caused a significant increase in PEFR.

The STN received high frequency stimulation in our patients. Although high frequency stimulation is suggested by some to be inhibitory to the nucleus as it creates the same clinical effect in PD as STN ablation, neurophysiological data suggests that it is in fact driving the nucleus in an excitatory fashion (Hamani, Brain 2004; 127:4-20, Hashimoto, J Neuroscience 2003; 23:1916-1923). This is reflected in its effect on autonomic performance whereby Thornton et al. produced heart rate and arterial blood pressure elevation using high frequency STN stimulation (Thornton, J Physiology 2002; 539(2):615-621).

Within the movement disorder cohorts, it could be suggested that the GPi group were all dystonic patients whereas the STN and PPN groups were comprised of PD patients. However, Thornton et al. demonstrated that neither high nor low frequency GPi stimulation in PD changed heart rate or mean arterial pressure whereas STN stimulation did (Thornton, J Physiology 2002; 539(2):615-621). Further, local field potential recordings from dystonic GPi nuclei during anticipation of exercise, during which central command mechanisms elevate cardiorespiratory variables, showed no increase in activity in contrast to the STN which increased beta and gamma band power (Green, J Physiology 2007; 578(2):605-612). Therefore there is evidence in both dystonia and Parkinson's patients that GPi behaves similarly with respect to cardiorespiratory control in the both diseases.

PPN stimulation for gait freezing, postural disability and akinesia in PD has been the focus of enormous interest within the neuroscience and neurosurgical communities within the last decade. Moro et al. postulate that its mode of action includes effects outside the motor system. They found no improvement one year after surgery in the objective motor assessments of the Unified Parkinson's Disease Rating Scale however there was a reduction in reported falls (Moro, Brain 2010; 133; 215-224). The improvement in rapid eye movement sleep demonstrated by Lim et al. with PPN stimulation (Lim, Annals Neurology 2009; 18:110-114) in addition to the results herein demonstrating an improvement in PEFR, an index of lung function, supports the notion of beneficial non-musculoskeletal effects from PPN stimulation.

### Electrode Mapping

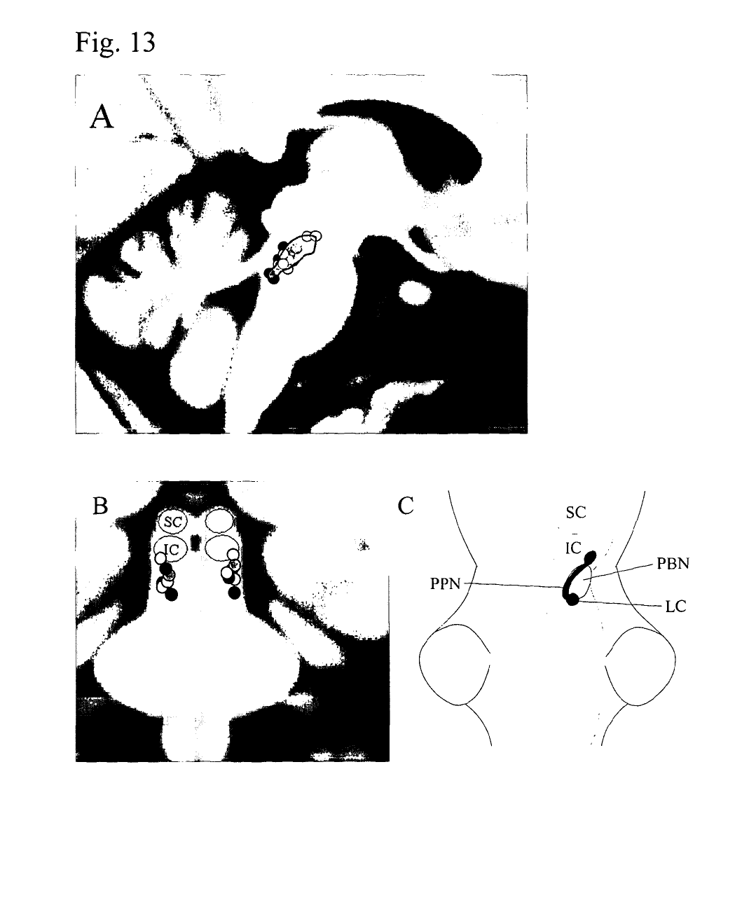

The electrode mapping reveals that the PPN electrodes also straddle, or are adjacent to, other important nuclei within the mesencephalic locomotor region/rostrodorsal pons. Given that the radius of electrical stimulation extends over 2 mm from the active electrode contacts (McIntyre, J Neurophysiology 2004; 91:1457-1469), it is possible that these other sites are being stimulated also. Most importantly, this includes the locus coeruleus (LC) and the parabrachial nuclei (PBN) which are recognised sites within the respiratory neurocircuitry of the brainstem (see FIG. 13). The LC is intimate to the caudal portion of the PPN, lying dorsally and infero-laterally to it. The LC is the major noradrenaline-containing nucleus of the brain (Berridge, Brain Research Reviews 2003; 42(1):33-84) and is the main noradrenergic structure implicated in AVPN inhibition (Haxhiu, Adv Med Exp Biol 2008; 605:469-474). Haxhiu et al. demonstrated in ferrets that LC stimulation causes relaxation of airway smooth muscle as a result of noradrenaline release and activation of alpha2A-adrenergic receptors on AVPNs, inhibiting their cholinergic outflow to the airway smooth muscle (Haxhiu, J Applied Physiology 2003; 94:1999-2009). The LC receives descending efferents from the PPN also, demonstrated in labelling studies in rats (Greene in Brain Cholinergic Systems, eds. Steriade M, Biesold D, Oxford University Press, United Kingdom, 1990). Therefore it is likely that the LC is activated either directly and/or indirectly by the stimulating macroelectrode in this study.

The medial and lateral PBN are also intimate to the PPN and lie beside its lateral border through most of its pontine length. Animal studies have implicated the PBN in the modulation of cardiovascular variables and the termination of inspiration whilst PBN destruction distorts the Hering-Breuer reflex (Gautier, Respiratory Physiology 1975; 23:71-85, Mraovitch, Brain Research 1982; 232:57-75) Motekaitis et al. chemically stimulated the PBN in anaesthetised cats causing a reduction in total lung resistance via a circuit requiring the caudal ventrolateral medulla and nucleus tractus solitarius (Motekaitis, J Applied Physio logy 1994; 76(4): 1712-1718, Motekaitis, J Applied Physiology 1996; 81(1):400-407).

It is therefore possible that deep brain stimulation of any one or all amongst the PPN, LC or PBN within the mesencephalic locomotor region/rostrodorsal pons may have accounted for the improvement in PEFR in our study. Further, it is possible that it is the structures beside the PPN, such as the LC and PBN, which are at least in part responsible for the clinical benefits seen in these patients. This study demonstrates that this mesencephalic/rostrodorsal pons region within the human brain contains a concentration of nuclei capable of facilitating airway smooth muscle relaxation.

The PAG is recognised to be integral to the fight or flight response. Stimulation of the PAG causes changes in cardiovascular variables, vocalisation and micturition (Bittencourt, Neuroscience 2004; 125:71-89, Carrive, Brain Research 1991; 541:206-15, McGaraughty, Brain Research 2004; 1009:223-7) via connections to medullary sites such as the rostral ventrolateral medulla which then projects to effectors of the sympathetic nervous system (Green, Experimental Physiology 2006; 93(9):102-1028). This study demonstrates that PAG stimulation also leads to improved PEFR, which further contributes to the fight or flight response, as gas exchange must be optimised during such stressful and metabolically-demanding activity. Relaxation of airway smooth muscle will increase gaseous flow between the atmosphere and alveoli, therefore increasing the intake of oxygenated air and the venting of carbon dioxide to facilitate further metabolically-demanding activity.

The PAG projects to the PBN (Holstege in The midbrain periaqueductal gray matter: functional anatomical and immunohistochemical organization (Depaulis A, Bandler R, eds), pp 239-265. New York: Plenum, 1991) and its activation may be the mechanism by which airway resistance was reduced by PAG stimulation in this study. Alternatively, as the PAG also projects to the PPN (Reese, Progress Neurobiology 1995; 42:105-133) this presents another possible route via which autonomic variables are augmented by PAG stimulation.

Within the medulla oblongata, the retrofacial nucleus, the nucleus tractus solitarius and the nucleus retroambiguus (NRA) are centres demonstrated in the cat to receive projections from the PAG (Bandler, Neuroscience Letters 1987; 74:1-6, Holstege, J Comparative Neurology 1989; 284:242-252, Sakamoto in Neural control of respiratory muscles (Miller A D, Bianchi A L, Bishop B P, eds), pp 249-258. Boca Raton, Fla.: CRC, 1996). These nuclei contain inspiratory neurons that drive, for example, the phrenic and external intercostal motorneurones (Duffin, J Physiology 1987; 390:415-431, Holstege, Progress Brain Research 1982; 57:145-175, Lipski, Brain Research 1983; 288:105-118) and in the case of the ventral NRA, the nucleus ambiguus as well (Holstege, J Comparative Neurology 1989; 284:242-252). The caudal NRA projects to motorneurones innervating internal intercostal, abdominal and pelvic floor muscles (Holstege in Progress in brain research, Vol 87 (Holstege G, ed), pp 307-421. Amsterdam: Elsevier, 1991) and therefore may make an important contribution to airflow during forced expiration with PAG stimulation.

### Example 2

### Further Experimental Data in Humans

In a separate experiment, an identical methodology was employed as above but patients were tested who had indwelling ACC and hypothalamus stimulators to treat pain syndromes (chronic neuropathic pain and cluster headache, respectively). Patients with motor thalamus stimulators were used as controls as this site is not implicated as part of the CAN.

TABLE 4
Summary of patient diagnoses, demographics and stimulation parameters in
the second experiment.

| Age (yrs)/ | | Stimulator | Stimulation Parameters (Voltage, Pulse |
|---|---|---|---|
| Sex | Diagnosis | Location | Width, Frequency, Electrode Contacts) |
| 55/M | Hemi-body pain | ACC Unilateral | 2.9 v, 170 μsec, 100 Hz |
| 41/F | Conus injury | ACC Bilateral | 3 v 270 μsec, 40 Hz |
| 73/M | Hemi-body pain | ACC Unilateral | 6 v, 300 μsec, 10 Hz |
| 62/M | Essential Tremor | MThal Unilateral | 3.5 v, 180 μsec, 130 Hz |
| 40/M | Functional Tremor | MThal Unilateral | 2.5 v, 150 μsec, 130 Hz |
| 66/F | Orthostatic Tremor | MThal Unilateral | 1.5 v, 90 μsec, 130 Hz |
| 61/F | Dystonic Tremor | MThal Unilateral | 3 v, 90 μsec, 130 Hz |
| 64/F | Parkinsonian Tremor | MThal Unilateral | 2.5 v, 150 μsec, 130 Hz |
| 61/M | Cluster Headache | PH Unilateral | 2 v, 60 μsec, 180 Hz |
| 56/M | Cluster Headache | PH Unilateral | 1.6 v, 60 μsec, 160 Hz |
| 48/F | Cluster Headache | PH Unilateral | 1.5 v, 90 μsec, 180 Hz |

(ACC = anterior cingulate cortex, MThal = motor thalamus (control), PH = posterior hypothalamus.)

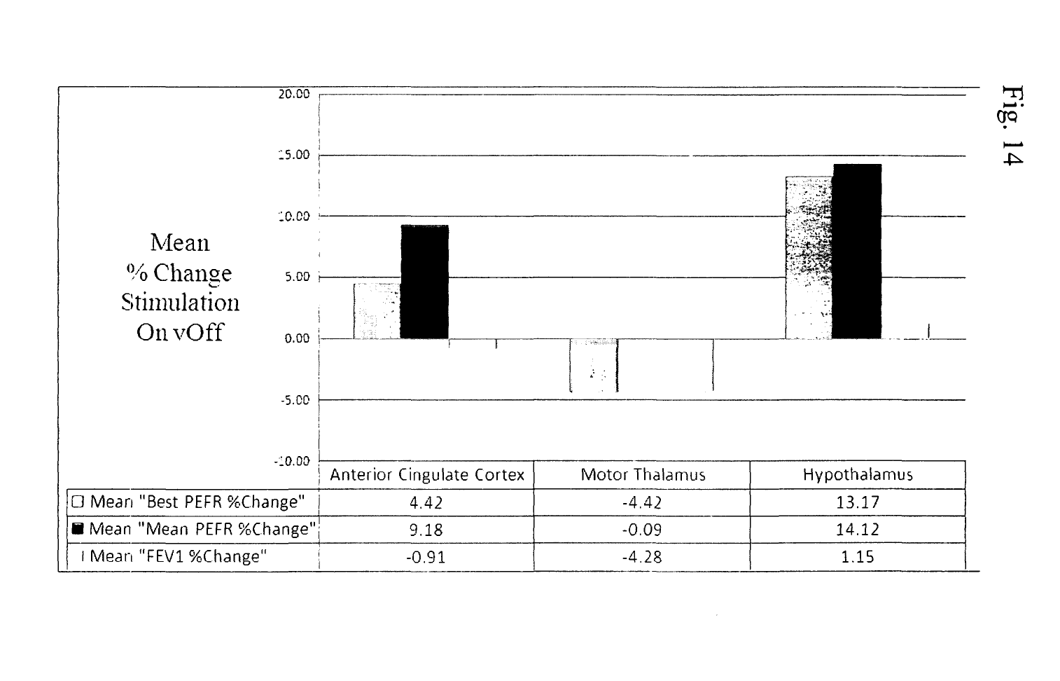

Three patients had ACC stimulation, two for hemi-body pain secondary to thalamic stroke and one for lower limb pain secondary to conus medullaris trauma; three patients had hypothalamic stimulators for cluster headache and five had motor thalamus stimulators for tremor (see Table 5). FIG. 14 shows that Anterior cingulate cortex and Hypothalamus stimulation improved PEFR whereas the motor thalamus (control) did not. Improvements in mean percentage PEFR was found in 2 out of 3 ACC subjects and all hypothalamic stimulation subjects, up to almost 30%. Mean percentage improvement in PEFR with ACC stimulation was 9.18% (range −1.6 to 23.6). Mean percentage improvement in PEFR with hypothalamic stimulation was 14.1% (range 1.6 to 29.9). Mean percentage improvement in PEFR with motor thalamus stimulation was −0.1% (range −10.3 to 17.2). Again, minimal change in FEV1 was seen after ACC and hypothalamic stimulation (−0.9% and 1.2%, respectively) with a decline of −4.4% with motor thalamus stimulation.

### Example 3

### Electrophysiological and Functional Evidence for the Role of the Pedunculopontine Nucleus in Respiratory Control

Introduction:

Neuronal oscillatory activity within subcortical brain has been shown to be an important factor in motor performance (Pogosyan A, Current Biology 2009; 19(19):1637-1641.). The PPN region is part of the reticular activating system and the mesencephalic locomotor region. PPN region stimulation is a novel therapy for gait freezing and postural instability in Parkinson's disease (PD). After administration of dopamine, PPN region oscillations synchronise within the 7-11 Hz band (Androulidakis A G, Experimental Neurology 2008; 211:59-66.). Dopamine has also been shown to improve upper airway calibre during forced respiratory manoeuvres in PD, a disease in which it is often compromised (Vincken W G, Chest 1989; 96(1):210-212.). The study described above has demonstrated that PPN stimulation can produce increases in PEFR. We therefore hypothesized that forced respiratory manoeuvres would be associated with a PPN region 7-11 Hz band synchronisation; and that low frequency electrical stimulation could improve indices of upper airway function.

Methods:

Patients with in-dwelling PPN region deep brain stimulators for PD were studied. Patients were trained to perform spirometry according to the European Respiratory Society guidelines. Patients performed 3 trials of maximal inspiration followed by forced expiration each with stimulation Off and On (at their regular therapeutic parameters). Conditions were randomised and patients blinded to stimulation settings. Patients received their regular anti-parkinsonian medication prior to testing. Indices of upper airway flow were recorded by spirometer: peak expiratory flow rate (PEFR), forced expiratory volume in 1 second (FEV1)/PEFR ratio, and maximal flow at 50% of forced vital capacity (FEF50). There was a ten-minute wash-out period between conditions. In patients with externalised electrodes, local field potentials (LFPs) were also recorded during the Off condition in a bipolar configuration and amplified 100,000 times and sampled at 1000 Hz. LFPs were decomposed into their constituent frequencies by fast Fourier transform allowing comparison between exertional manoeuvres of maximal inspiration and forced expiration and resting breathing.

Results:

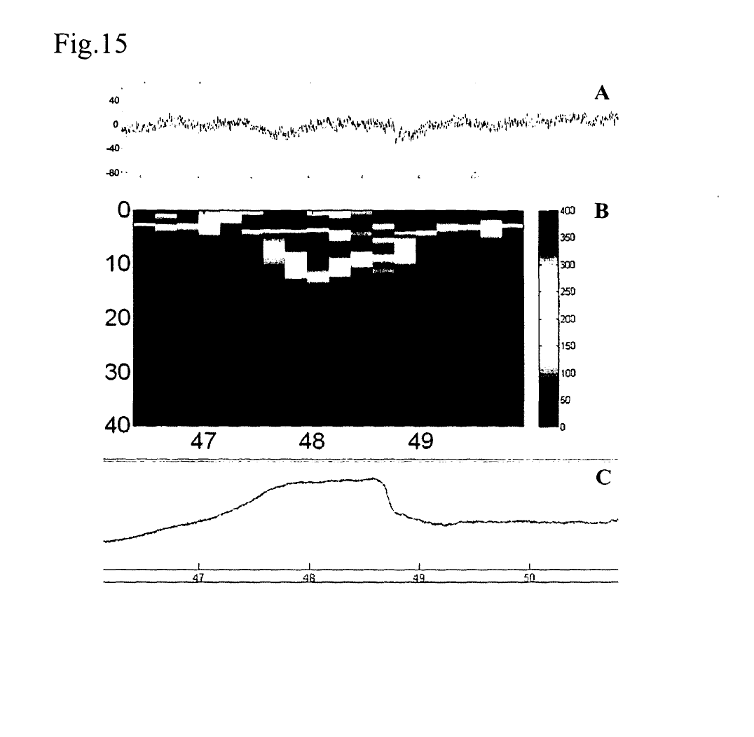

Nine patients were studied. LFPs were recorded in 7 cases. Mean PPN LFP power increased significantly within the 7-11 Hz Alpha band during exertional respiratory manoeuvres (1.63 μV2/Hz (SE+0.16 μV2/Hz)) compared to resting breathing (0.77 μV2/Hz (SE+0.16 μV2/Hz)); z=−2.197, df=6, p=0.028 (see FIG. 15). PEFR increased significantly by a mean of 15.8% with stimulation, from 6.41 L/s (SE+0.63 L/s) in the Off state to 7.5 L/s (SE+0.65 L/s) in the On state (z=−2.666, df=8, p=0.032). Mean FEV1/PEFR ratio improved from 7.21 ml/L/min (SE+0.45) to 6.75 ml/L/min (SE+0.42) which was statistically significant (z=−2.666, df=8, p=0.024). Mean FEF50 increased from 3.45 L/s (SE+0.36 L/s) to 3.83 L/s (SE+0.5 L/s) with stimulation although this did not reach statistical significance (p=0.063). Percentage improvement in PEFR was strongly correlated to proximity of stimulating electrode contact to the mesencephalic locomotor region in the rostral PPN (r=0.814, n=9, p=0.008).

Conclusions:

There was a synchronisation of PPN region oscillatory activity in the 7-11 Hz band during forced respiratory manoeuvres. Further, electrically stimulating the PPN region at the same site in these patients at low frequency produced improved performance indices during these manoeuvres, particularly relating to upper airway function. This may confer benefit for patients with upper airway dysfunction in PD or in other upper airway diseases including obstructive sleep apnoea. Thus the PPN region, particularly its more rostral portion, appears to be an important site in producing exertional respiration as its cells are electrically synchronised during these manoeuvres, and electrical stimulation confers improved lung function.

### Example 4

### Disruption of Anterior Cingulate Cortex Function by Neurosurgery Reduces Dyspnoea in Humans with Terminal Lung Disease

The neural circuitry within the brain which facilitates the perception of dyspnoea has been examined in imaging studies. These implicate the anterior cingulate cortex, the insula and the amygdala within this circuitry (for a review see Herigstad M, Respiratory Medicine 2011; 105(6):809-817). We studied the degree of dyspnoea in patients with terminal mesothelioma after performing radio frequency lesioning of the anterior cingulate cortex.

### Methods

Two patients with terminal mesothelioma and thoracic pain underwent anterior cingulate radio frequency ablation bilaterally for pain relief. Pre- and post-operative assessments were performed. The—Were you short of breath∥ component of the European Organization for Research and treatment of Cancer Quality of life questionnaire (EORTC QLQ C-30) were recorded out of a maximum severity of 4 (where 1=Not at all; 2=A little; 3=Quite a bit; 4=Very much). The “Have you had (chest) pain?” component was also recorded out of 4 to control for simply an improvement in pain relief explaining any change in dyspnoea. Patients rated on a visual analogue scale (0-100) the quantity of “Breathlessness today” and “How much has the breathlessness bothered you today”.

### Results

Improvements in all indices were recorded at one month after surgery. “Were you short of breath?” outcome improved from 3 to 2 in both patients. “Breathlessness today?” and “How much has the breathlessness bothered you today?” outcomes were available in Patient 1 and improved from 50/100 to 21/100 and 49/100 to 20/100, respectively (see figure Table 5). Although the pain index “Have you had (chest) pain today?” improved in one patient from 3 to 2, it worsened in the other patient form 3 to 3.5, suggesting that the dyspnoea relief is independent from pain amelioration and therefore is mediated by a different pathway. Over longer follow-up in Patient 1, the pain and dyspnoea scores increased again reaching 56/100 in both “Breathlessness today?” and “How much has the breathlessness bothered you today?” outcomes.

TABLE 5
Outcome variables at one-month follow-up after anterior cingulate cortex
lesioning.

| | Patient 1 | | Patient 2 | |
|---|---|---|---|---|
| | Pre-op | Post-op | Pre-op | Post-op |
| Were you short of breath? | 3 | 2 | 3 | 2 |
| Have you had (chest) pain | 3 | 2 | 3 | 3.5 |
| Breathlessness today | 50 | 21 | — | — |
| How much has this breathless | 49 | 20 | — | — |
| bothered you today | | | | |

### CONCLUSIONS

Radio frequency lesioning of the anterior cingulate cortex improves dyspnoea scores at one-month follow-up. As the effect of lesioning is believed to be functionally equivalent to electrical stimulation (as the same clinical effect results for example after thalamic lesioning and thalamic electrical stimulation in humans with tremor), and further, that electrical stimulation of the anterior cingulate cortex is known to improve pain perception in patients with refractory neuropathic pain syndromes (Spooner J, J Neurosurgery 2007; 107:169-172) in a similar fashion to cingulate lesioning, this study provides evidence that deep brain stimulation of the anterior cingulate cortex could improve the debilitating symptom dyspnoea. Longer follow-up after lesioning showed a reduction in dyspnoea amelioration in one patient. This may reflect the progressive nature of mesothelioma or alternatively the development of tolerance after lesioning. In either case deep brain stimulation could provide greater therapeutic opposition to this as it parameters can be varied, allowing modification and titration of stimulation settings over time.

1. A method of influencing bronchoconstriction in a mammal comprising applying a stimulation in one or more regions of the brain of the mammal.

2. A method of treating a respiratory disease or sleep apnea in a mammal comprising applying a stimulation in one or more regions of the brain of the mammal.

3. A method according to claim 1 wherein the mammal has a respiratory disease or sleep apnea.

4. A method according to claim 2 wherein the respiratory disease is an obstructive lung disease, reversible airways disease, asthma, chronic obstructive pulmonary disease (COPD), emphysema, bronchitis, Ondine's curse, lung cancer, tuberculosis or a lung disease where shortness of breath is a chronic symptom.

5. A method according to claim 1 wherein the stimulation causes bronchodilation.

6. A method according to claim 1 wherein the stimulation is deep brain stimulation.

7. A method according to claim 1 wherein the stimulation includes at least one member selected from the group consisting of an electrical stimulation, a magnetic stimulation, an electromagnetic stimulation, a radiofrequency stimulation, a biological tissue implantation, a thermal stimulation, an ultrasound stimulation and a chemical stimulation.

8. A method according to claim 1 wherein the one or more regions are selected from the periaqueductal grey matter of the midbrain (PAG), the subthalamic nucleus (STN), the pedunculopontine nucleus (PPN), the locus coeruleus (LC), the parabrachial nuclei (PBN), the hypothalamus, the anterior cingulate cortex (ACC), the insula cortex and the amygdala.

9. A method according to claim 1 wherein applying the stimulation includes generating a voltage differential between at least two electrodes of between about −10V and about +10V with a frequency of between about 0.1 Hz and about 1 kHz, preferably between about 10 Hz and 130 Hz, and a pulse width of 5 μsecs and 1000 μsecs.

10. A method according to claim 1 further including feeding back a metric representative of bronchoconstriction or blood oxygenation in an automated manner, or enabling feedback of a metric representative of bronchoconstriction, respiratory function including respiratory rate, or blood oxygenation in a manual manner, and adjusting the stimulation in response to the metric.

11. An apparatus for influencing bronchoconstriction in a mammal, comprising: a sensor detecting the extent of bronchoconstriction or derangement of respiratory activity or gas exchange in the mammal; a processor in communication with the sensor and generating a control signal based on the extent of bronchoconstriction or derangement of respiratory activity or gas exchange; a signal generator in communication with the processor generating a stimulation signal based on the control signal; and an electrode including at least two conductors in contact with a region of the brain that stimulates the region as a function of the stimulation signal in a manner influencing bronchoconstriction in the mammal.

12. An apparatus for influencing blood oxygenation in a mammal, comprising: a sensor detecting the level of oxygen in the blood of the mammal; a processor in communication with the sensor and generating a control signal based on the level of oxygen in the blood of the mammal; a signal generator in communication with the processor generating a stimulation signal based on the control signal; and an electrode including at least two conductors in contact with a region of the brain that stimulates the region as a function of the stimulation signal in a manner influencing blood oxygenation in the mammal.

13. An apparatus for stimulating a region in a human brain, comprising: a signal generator adapted to generate a signal; and at least one electrode disposed in a region of a brain in a human subject adapted to produce an output as a function of the signal to stimulate the region in a manner influencing bronchoconstriction or blood oxygenation in the human subject.

14. An apparatus according to claim 13 wherein the signal generator is coupled to a receiver configured to receive stimulation parameters used for applying the stimulation by at least one member selected from the group consisting of a radio frequency signal, electrical signal, and optical signal.
